SEQUENCE LISTING (42 sequences)

SEQ ID NO: 1 - DNA, 21 bp, Artificial
  Synthetic
cgccggcattctacaggacat

SEQ ID NO: 2 - DNA, 21 bp, Artificial
  Synthetic
cattctccctcctcttcggtc

SEQ ID NO: 3 - DNA, 21 bp, Artificial
  Synthetic
aagagaagtattcaagggata

SEQ ID NO: 4 - DNA, 21 bp, Artificial
  Synthetic
atccaacaaggcactgaccat

SEQ ID NO: 5 - DNA, 21 bp, Artificial
  Synthetic
gccggcattctacagggatac

SEQ ID NO: 6 - DNA, 24 bp, Artificial
  Synthetic
gtgggaatgatgaaaggtggcttt

SEQ ID NO: 7 - DNA, 21 bp, Artificial
  Synthetic
gcggaaccgagatgatgtagc

SEQ ID NO: 8 - DNA, 19 bp, Artificial
  Synthetic
caagcccgctcatgatcaa

SEQ ID NO: 9 - DNA, 21 bp, Artificial
  Synthetic
attttccctggttcctggcac

SEQ ID NO: 10 - DNA, 23 bp, Artificial
  Synthetic
agctggttcacatgatcaactgg

SEQ ID NO: 11 - DNA, 21 bp, Artificial
  Synthetic
cagaaatgtgtacactcctgt

SEQ ID NO: 12 - DNA, 22 bp, Artificial
  Synthetic
ctgttttgctcctaacttgctc

SEQ ID NO: 13 - DNA, 21 bp, Artificial
  Synthetic
ctccatgcctttgttacagaa

SEQ ID NO: 14 - DNA, 24 bp, Artificial
  Synthetic
ctgatcatggagggtcaaatccac

SEQ ID NO: 15 - DNA, 25 bp, Artificial
  Synthetic
acacaccttacctgtaaacaggaca

SEQ ID NO: 16 - DNA, 18 bp, Artificial
  Synthetic
gctgctcgtcggcacttc

SEQ ID NO: 17 - DNA, 23 bp, Artificial
  Synthetic
tttaaaagaagcattcaaggctc

SEQ ID NO: 18 - DNA, 20 bp, Artificial
  Synthetic
aactccttgtcggccaactt

SEQ ID NO: 19 - DNA, 21 bp, Artificial
  Synthetic
gaatggtgaagtgtggctttg

SEQ ID NO: 20 - DNA, 21 bp, Artificial
  Synthetic
atcatggccttgacacagaga

SEQ ID NO: 21 - DNA, 18 bp, Artificial
  Synthetic
cggcaaggccaagagaag

SEQ ID NO: 22 - DNA, 21 bp, Artificial
  Synthetic
gcatttcccctcatcccggga

SEQ ID NO: 23 - DNA, 21 bp, Artificial
  Synthetic
catgatgatgtccctgaccaa

SEQ ID NO: 24 - DNA, 21 bp, Artificial
  Synthetic
gaccagagggtacatacctgt

SEQ ID NO: 25 - DNA, 21 bp, Artificial
  Synthetic
ccagagggtacataccgggaa

SEQ ID NO: 26 - DNA, 20 bp, Artificial
  Synthetic
tttgggtgattgccaagagc

SEQ ID NO: 27 - DNA, 18 bp, Artificial
  Synthetic
agcacgtgggcattcagc

SEQ ID NO: 28 - DNA, 20 bp, Artificial
  Synthetic
gtcactgctccatcgttgct

SEQ ID NO: 29 - DNA, 22 bp, Artificial
  Synthetic
caggcctcaaaagatctgcttc

SEQ ID NO: 30 - DNA, 24 bp, Artificial
  Synthetic
gtctgggttttatatcgtctgcaa

SEQ ID NO: 31 - DNA, 23 bp, Artificial
  Synthetic
cacttttcccaaatcacttcacc

SEQ ID NO: 32 - DNA, 23 bp, Artificial
  Synthetic
tgtccagccaccaacagtgcacc

SEQ ID NO: 33 - DNA, 23 bp, Artificial
  Synthetic
agaacagcctggccttgtccctg

SEQ ID NO: 34 - DNA, 23 bp, Artificial
  Synthetic
tggtggagatcttcgacatgctg

SEQ ID NO: 35 - DNA, 22 bp, Artificial
  Synthetic
tccatggagcacccagtgaagc

SEQ ID NO: 36 - DNA, 21 bp, Artificial
  Synthetic
tgggtaccgccttgtgcggag

SEQ ID NO: 37 - DNA, 25 bp, Artificial
  Synthetic
tagtgctcaccctcctggaggctga

SEQ ID NO: 38 - DNA, 27 bp, Artificial
  Synthetic
aggtgttaatgatggggctgatgtggc

SEQ ID NO: 39 - DNA, 22 bp, Artificial
  Synthetic
cctcccagcagcaatccatgcg

SEQ ID NO: 40 - DNA, 19 bp, Artificial
  Synthetic
ttccaggagcgagatccct

SEQ ID NO: 41 - DNA, 23 bp, Artificial
  Synthetic
ggctgttgtcatacttctcatgg

SEQ ID NO: 42 - DNA, 22 bp, Artificial
  Synthetic
tgctggcgctgagtacgtcgtg

(19) United States
(12) Patent Application Publication
(10) Pub. No.: US 2004/0058345 A1
(43) Pub. Date: Mar. 25, 2004

(54) Method of generating an expression profile of estrogen receptor isoforms and related methods

(75) Inventor: Indra Poola, Gaithersburg, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Silbiotech, Gaithersburg, MD

(21) Appl. No.: 10/375,597

(22) Filed: Feb. 27, 2003

Related U.S. Application Data
(60) Provisional application No. 60/361,072, filed on Feb. 27, 2002.
(60) Provisional application No. 60/416,956, filed on Oct. 4, 2002.

(51) Int. Cl. C12Q 1/68; C07H 21/04; C12P 19/34

(52) U.S. Cl. 435/6; 435/91.2; 536/23.2

(57) ABSTRACT

A method of amplifying and quantifying a mRNA transcript of the α or β isoform of the ER; a method of generating an expression profile of mRNA transcripts of the α or β isoform of the ER, wherein the mRNA transcripts comprise wild-type or alternatively spliced exons, comprising reverse-transcribing the mRNA transcript, amplifying the resulting cDNA using a targeted primer and a partner primer, contacting the resulting amplicons with a probe, detecting the probe, and quantifying the detected probe; isolated oligonucleotides to be used as a targeted primer, partner primer, or probe; a method of determining a hyperproliferative condition or a predisposition to a hyperproliferative condition, such as breast cancer or a risk for breast cancer; a method of screening candidate therapeutic treatments for an ER-sensitive condition; and a method of prognosticating response of a mammal to a therapeutic treatment of breast cancer; and other methods.

### FIELD OF THE INVENTION

This invention pertains to isolated oligonucleotides and the use of these and other isolated oligonucleotides in the amplification of mRNA transcripts comprising wild-type or alternatively spliced exons of the α or β isoform of the estrogen receptor (ER) genes, which, in turn, is used in conjunction with methods of generating cRNA of a GAPDH or of an α or β isoform of the ER and methods of generating a standard curve for generating an expression profile of alternatively spliced isoforms of the ER, which, in turn, is used for determining a hyperproliferative condition, or a predisposition to a hyperproliferative condition, such as breast cancer, for prognosticating response to a therapeutic treatment of breast cancer, and for screening candidate therapeutic treatments for an ER-sensitive condition.

### BACKGROUND OF THE INVENTION

Breast cancer is the second leading cause of cancer-related deaths for women in the United States, killing approximately 40,000 women and inflicting about 203,500 women in the year 2001 alone (Jamal et al., CA Cancer J. Clin. 52: 23-47 (2002)). Epidemiological, experimental, and clinical evidence all corroborate that the endogeneous hormone, estrogen, plays a major role in the genesis and advancement of breast cancers (Henderson et al., Cancer Res. 48:246-252 (1988)). Because estrogens simulate the growth of breast cancer cells, anti-estrogens, which inhibit the mitogenic effects of the endogeneous hormones, have been a major focus of breast cancer treatments (Simon et al., J Natl. Cancer Instit. 73:313-321 (1984)).

Anti-estrogens compete with endogeneous estrogens for binding sites on the ERα, thereby inhibiting the gene transcription that is activated upon the binding of estrogen-ERα complexes to the promoter region of the genes. For more than ten years, anti-estrogen therapy has successfully treated women diagnosed with breast cancer. In particular, Tamoxifen has proven to restrict the growth of breast tumors, thereby extending the lives of thousands of women. However, not all breast cancer patients have responded favorably to Tamoxifen therapy. The efficacy of this treatment has been found to correlate with the presence of the ERα in tumor tissue. Consequently, only patients who express the ERα in tumor tissue as determined by immunohistochemistry are chosen for Tamoxifen therapy (Osborn et al., Cancer 46:2884-2888 (1980); Thorpe et al., Acta Oncol. 27:1-19 (1988)). Other factors must be contributing to the effectiveness of this therapy, however, since only half of the tumors, which express the ERα, are responsive to anti-estrogen therapy. Although altered expression patterns of specific growth factors and oncogenes have been suggested to be factors in the success of anti-estrogen therapy, the most important factor appears to be the expression of different ER isoforms.

A number of ER isoforms are now known to exist, in addition to the classical ER, ERα. These include ERβ, the twenty alternatively spliced variants of ERα and the fourteen alternatively spliced variants of ERβ (McGuire et al., Mol. Endocrinol. 5:1571-1577 (1991); Fuqua et al., Cancer Res. 51:105-109 (1991); Castles et al., Cancer Res. 53:593-9 (1993); Leygue et al., Cancer Res. 56:4324-4327 (1996); Poola et al., J. Steroid Biochem. Mol. Biol. 72:249-258 (2000); Pfeffer et al., Cancer Res., 55:2158-2165 (1995); Gotteland et al., Mol. Cell Endocrinol. 112:1-13 (1995); Speirs et al., J. Clin. Endocrinol. Metab. 85:1601-5 (2000); Lu et al., Mol. Cell. Endocrin. 7:205-211 (1998); Vladusic et al., Cancer Res. 58:210-4 (1998); Fuqua et al., Cancer Res. 59:5425-5428 (1999); Moore et al., Bichem. Biophys. Res. Comm. 247: 75-78 (1998); Poola et al., FEBS Letters 516: 133-138 (2002)). ERα and ERβ are highly homologous but are encoded by two different genes in two different chromosomes (Green et al., Nature 320: 134-139 (1986) and Enmark et al., J. Clin. Endocrin. Metab. 82: 4258-4265 (1997)). They differ in their ability to bind estrogen and anti-estrogens as well as in their ability to activate gene transcription. For example, ERβ has been shown to exhibit a lower affinity for Tamoxifen (Kuiper et al., Endocrinology 138:863-869 (1997); Paech et al., Science 277:1508-1510 (1997); and Cowley et al., J. Steroid Biochem. Mol. Biol. 69:165-75 (1999)). Accordingly, Tamoxifen resistance correlates with elevated levels of ERβ expression (Speirs et al., Cancer Res. 59:5421-5424 (1999)). The ERα3Δ isoform, which has exon 3 of the ERα gene deleted, has the same affinity for estrogens and anti-estrogens as wild-type ERα, but exhibits a dominant negative effect in that it inhibits gene transcription by the wild-type receptor. Although the exon 5 deletion mutant of ERα, ERα5Δ, can activate gene transcription to similar extents as the wild-type receptor, it does so constitutively, inducing the transcription of genes even in the absence of estrogen stimulation (Bollig et al., Mol. Endocrinology 14: 634-649 (2000)).

Since breast tumors are heterogenous in their composition of the various ER isoforms (McGuire et al. (1991), supra; Fuqua et al. (1991), supra; Castles et al. (1993), supra; Leygue et al. (1996), supra; Poola et al. (2000), supra; Pfeffer et al. (1995), supra; Gottelend et al. (1995), supra; Speirs et al. (2000), supra; Lu et al. (1998), supra; Vladusic et al. (1998), supra; Fuqua et al. (1999), supra; Poola et al. (2001), supra; Poola et al., J. Steroid Biochem. Mol. Biol. 78: 459-469 (2001); Poola et al., Cancer 94: 615-623 (2002a) and Poola et al., J. Steroid Biochem. Mol. Biol, 82, 169-179 (2002b)) and because the ER isoforms differ with respect to the affinities for anti-estrogens, it logically follows that the relative expression level of each receptor in tumor tissues will influence the effects that anti-estrogens have on these tissues. There exists a need, therefore, to quantify precisely all isoforms of the ER on tumor tissues for prognosticating a response of a patient to anti-estrogen therapy.

Currently, immunohistochemistry using antibodies specific to the N-terminal region of the classical ER is the method for determining the expression level of the ER on tumor tissue. However, this method cannot distinguish all of the known isoforms of the ER, since the antibodies used in this method cross-react with more than one ER isoform. Also, because immunohistochemistry is not sensitive enough to detect low levels of the ER, a second method, such as polymerase chain reaction (PCR), is often times employed to confirm the presence or absence of the ER in tumor tissue. Furthermore, immunohistochemistry requires relatively large tumor samples for the detection of multiple ER isoforms. Moreover, the assessment of multiple isoforms is expensive. Finally, immunohistochemistry does not allow for a quantitative analysis of ER isoform expression. Therefore, a highly sensitive, rapid, cost-effective clinical method that can precisely detect and quantify every known ER isoform from a small amount of tumor tissue is needed in the art.

The present invention provides such a method. This and other objects and advantages of the invention, as well as additional inventive features, will be apparent from the description of the invention provided herein.

### BRIEF SUMMARY OF THE INVENTION

The present invention provides an isolated oligonucleotide consisting essentially of the nucleotide sequence selected from the group consisting of SEQ ID NO: 8, SEQ ID NOs: 15-27, SEQ ID NO: 40, and SEQ ID NO: 41, as well as an isolated oligonucleotide consisting essentially of the nucleotide sequence selected from the group consisting of SEQ ID NOs: 32-39 and SEQ ID NO: 42.

Also provided is a method of amplifying a mRNA transcript of the β isoform of the ER, wherein the mRNA transcript comprises alternatively spliced exons. The method comprises reverse-transcribing the mRNA transcript and amplifying the resulting cDNA using a pair of primers comprising a targeted primer and a partner primer. The targeted primer is an isolated oligonucleotide comprising two parts, A and B, of which A is about 16 to about 25 nucleotides in length and comprises the sequence of a first exon, wherein the sequence of the first exon is immediately adjacent to an alternate splice junction, which is located between the first exon and a second exon. B, which is 3′ to A, is (a) at most 8 nucleotides in length, (b) comprises the sequence of a second exon, wherein the sequence of the second exon is immediately adjacent to the alternate splice junction, and (c) comprises at least 3 nucleotides that are unique to the second exon. The partner primer is an isolated oligonucleotide about 20 to about 25 nucleotides in length and comprises the sequence of or the sequence that is complementary to any sequence found either within about 600 nucleotides upstream of the alternate splice junction or within about 600 nucleotides downstream of the alternate splice junction.

Further provided is a method of generating an expression profile of mRNA transcripts of the β isoform of the ER, wherein the mRNA transcripts comprise alternatively spliced exons. The method comprises amplifying a mRNA transcript of the β isoform of the ER according to the above method, contacting the resulting amplicons with a detectably-labeled probe, detecting the detectably-labeled probe, and quantifying the detectably-labeled probe detected. The number of copies of a particular mRNA transcript of the β isoform of the ER is determined by this method.

Still further provided is a method of amplifying a mRNA transcript of the α isoform of the ER, wherein the mRNA transcript comprises alternatively spliced exons. The method comprises reverse-transcribing the mRNA transcript and amplifying the resulting cDNA using a pair of primers comprising a targeted primer and a partner primer selected from the group consisting of SEQ ID NO: 1 and SEQ ID NO: 2, SEQ ID NO: 3 and SEQ ID NO: 4, SEQ ID NO: 5 and SEQ ID NO: 4, SEQ ID NO: 6 and SEQ ID NO: 7, SEQ ID NO: 8 and SEQ ID NO: 9, SEQ ID NO: 10 and SEQ ID NO: 11, and SEQ ID NO: 12 and SEQ ID NO: 13.

The present invention further provides a method of generating an expression profile of mRNA transcripts of the α isoform of the ER, wherein the mRNA transcripts comprise alternatively spliced exons. The method comprises amplifying a mRNA transcript of the α isoform of the ER according to the above method, contacting the resulting amplicons with a detectably-labeled probe, detecting the detectably-labeled probe, and quantifying the detectably-labeled probe detected. Through this method, the number of copies of a particular mRNA transcript of the α isoform of the ER is determined.

A method of generating an expression profile of a mRNA transcript of a full-length, wild-type α or β isoform of the ER is further provided by the present invention. The method comprises reverse transcribing the mRNA transcript, amplifying the resulting cDNA using a pair of primers comprising SEQ ID NO: 8 and SEQ ID NO: 14, SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, or SEQ ID NO: 26 AND SEQ ID NO: 31, contacting the resulting amplicons with a detectably-labeled probe, detecting the detactably-labeled probe, and quantifying the detectably-labeled probed detected. Upon this method, the number of copies of the full-length, wild-type mRNA transcript of the α or β isoform of the ER is determined.

Also provided by the present invention is a method of generating a standard curve. The standard curve correlates a number of copies of in vitro-transcribed cRNA transcripts with a quantity of a detectably-labeled probe detected. The method comprises in vitro transcribing RNA from a DNA plasmid, which encodes GAPDH, the wild-type protein of the α or β ER, an alternatively-spliced isoform of the α or β ER, or a fragment of either of the foregoing, wherein, when the fragment is that of an alternatively spliced isoform of the α or β ER, the fragment comprises the alternative splice junction. The method further comprises reverse-transcribing the resulting cRNA and amplifying the resulting cDNA using the pair of primers that is used to amplify GAPDH, the wild-type protein of the α or β ER, the alternatively-spliced isoform of the α or β ER, or a fragment of either of the foregoing encoded in the DNA plasmid. The pair of primers is selected from the group consisting of SEQ ID NO: 40 and SEQ ID NO: 41; SEQ ID NO: 1 and SEQ ID NO: 2; SEQ ID NO: 3 and SEQ ID NO: 4; SEQ ID NO: 5 and SEQ ID NO: 4; SEQ ID NO: 6 and SEQ ID NO: 7; SEQ ID NO: 8 and SEQ ID NO: 9; SEQ ID NO: 10 and SEQ ID NO: 11; SEQ ID NO: 12 and SEQ ID NO: 13; SEQ ID NO: 8 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 21 and SEQ ID NO: 25; SEQ ID NO: 26 and SEQ ID NO: 27; SEQ ID NO: 26 and SEQ ID NO: 28; SEQ ID NO: 26 and SEQ ID NO: 29; SEQ ID NO: 26 and SEQ ID NO: 30; and SEQ ID NO: 26 and SEQ ID NO: 31. The method further comprises contacting the resulting amplified products with a detectably-labeled probe, detecting the detectably-labeled probe, quantifying the detectably-labeled probe detected, and repeating the steps from reverse-transcribing through quantifying at least 6 times, wherein the amount of cRNA used in the reverse-transcription is different each time. Through this method a standard curve correlating a number of copies of in vitro-transcribed cRNA with a quantity of detectably-labeled probe detected is generated.

The present invention also provides a method of generating cRNA of a GAPDH or of an α or β isoform of the ER. The method comprises cloning a fragment of a coding sequence of the GAPDH or of the α or β isoform of the ER into a pCR®2.1-TOPO vector Gibco-BRL Life Technologies, Gaithersburg, Md.), wherein the fragment is at least about 500 base pairs in length, and then linearizing the resulting vector with the restriction enzyme BamHI. The method further comprises using T7 RNA polymerase to generate cRNA and then cleaving the vector with DNA-free™DNAse. Through this method, cRNA for the GAPDH or the α or β isoform of the ER is generated.

The present invention further provides a different method of generating cRNA in the case that the isoform is ERα5Δ. In this method, a fragment of a coding sequence of the ERα5Δ isoform is cloned into a pCR®2.1-TOPO vector. However, the restriction enzyme EcoRI is used to digest the resulting vector to obtain a second fragment containing the fragment of a ERα5Δ coding sequence. The resulting second fragment is subcloned into a pSG vector (Stratagene, La Jolla, Calif.), which is then digested with the restriction enzyme BglII. T7 RNA polymerase is used to generated cRNA, and the vector is cleaved with DNA-free™DNAse.

A method of determining breast cancer or a risk for breast cancer in a mammal is further provided by the present invention. The method comprises comparing the expression profile of an ER-containing sample of the mammal with the expression profile of a control standard, wherein the expression profiles have been obtained in accordance with any of the above methods. A difference in the expression profile of the mammal in comparison to the expression profile of the control standard is indicative of breast cancer or risk for breast cancer in the mammal.

Still further provided by the present invention is a method of prognosticating response of a mammal to a therapeutic treatment of breast cancer in the mammal. The method comprises comparing the expression profiles of ER-containing samples of the mammal obtained over the course of therapeutic treatment of the mammal, wherein the expression profiles are obtained in accordance with any of the above methods.

A method of determining a hyperproliferative condition or a predisposition to a hyperproliferative condition in a mammal is further provided by the present invention. The method comprises comparing the expression profile of an ER-containing sample of the mammal with the expression profile of a control standard, wherein the expression profiles are obtained in accordance with any of the above methods. A difference in the expression profile of the mammal in comparison to the expression profile of the control standard is indicative of a hyperproliferative condition or a predisposition to a hyperproliferative condition in the mammal.

Further provided by the present invention is a method of screening candidate therapeutic treatments for an ER-sensitive condition. The method comprises comparing the expression profile of an ER-containing sample before treatment with the expression profile of the ER-containing sample after treatment, wherein the expression profiles are obtained in accordance with any of the above methods.

### BRIEF DESCRIPTION OF THE DRAWINGS

FIG. 1 represents a table of nucleotide sequences (5′→3′ when read from left to right) of specific targeted primers, partner primers, and probes of the present invention.

FIG. 2 represents a table specifying which primers and probes of the present invention are to be used to detect a particular ER isoform or GAPDH.

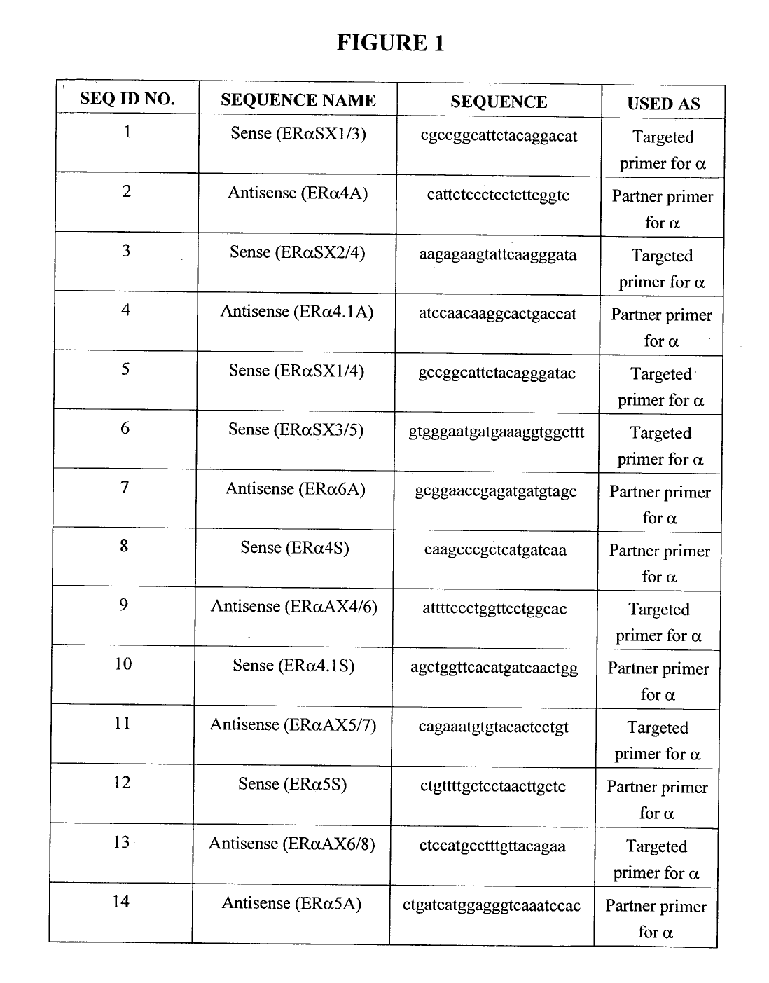

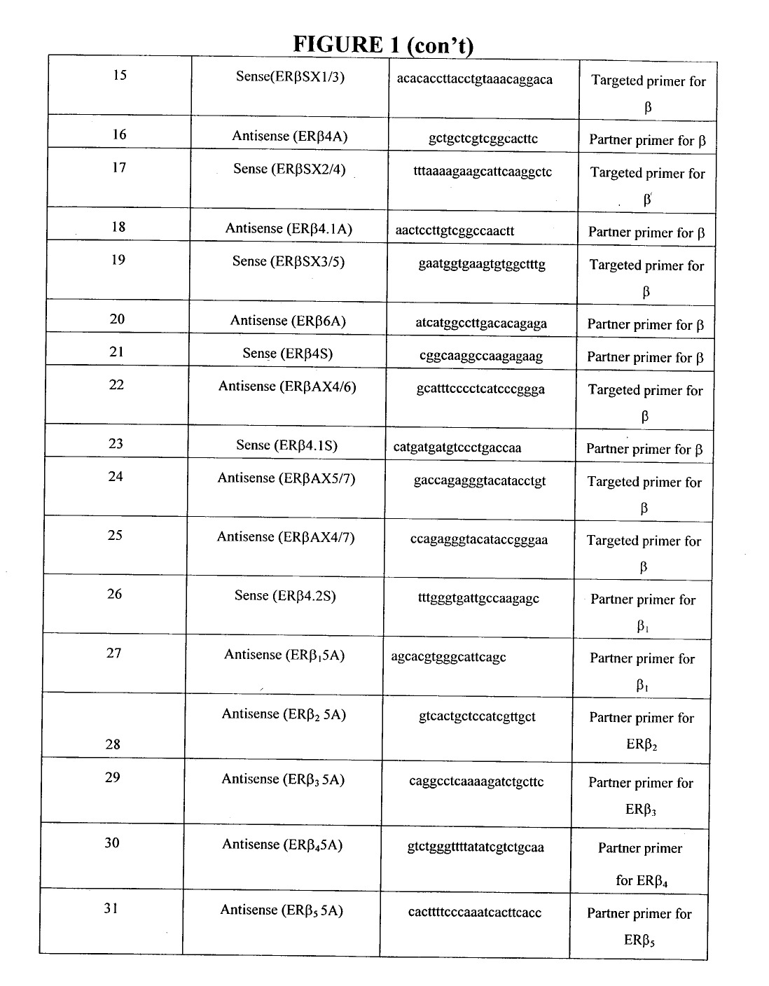

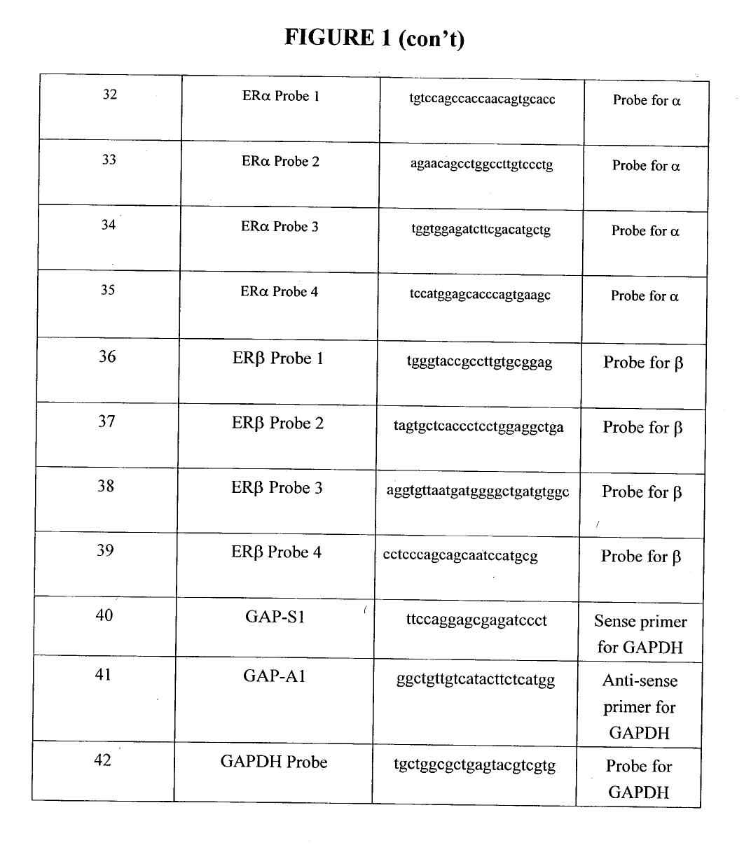

### DETAILED DESCRIPTION OF THE INVENTION

The present invention provides an isolated oligonucleotide consisting essentially of the nucleotide sequence selected from the group consisting of SEQ ID NO: 8, SEQ ID NOs: 15-27, SEQ ID NO: 40, and SEQ ID NO: 41, which are specific primers to be used in the methods described herein, as well as an isolated oligonucleotide consisting essentially of the nucleotide sequence selected from the group consisting of SEQ ID NOS: 32-39 and SEQ ID NO: 42, which are specific probes to be used in the methods described herein. The term “isolated” as used herein is defined as having been removed from its natural environment. The term “oligonucleotide” as used herein is defined as a polymer of DNA or RNA, (i.e., a polynucleotide), which can be single-stranded or double-stranded, synthesized or obtained from natural sources, and which can contain natural, non-natural or altered nucleotides. With respect to the isolated oligonucleotides of the present invention, it is preferred that no insertions, deletions, inversions, and/or substitutions are present in the oligonucleotide. However, it may be suitable in some instances for the isolated oligonucleotides of the present invention to comprise one or more insertions, deletions, and/or substitutions. It is, furthermore, preferred that the isolated oligonucleotides of the present invention are synthesized, single-stranded polymers of DNA.

A method of amplifying a mRNA transcript of the β isoform of the ER, wherein said mRNA transcript comprises alternatively spliced exons, is further provided by the present invention. The method comprises reverse transcribing the mRNA transcript and amplifying the resulting cDNA using a pair of primers comprising a targeted primer and a partner primer. The targeted primer is an isolated oligonucleotide comprising two parts, A and B, of which A is about 16 to about 25 nucleotides in length and comprises the sequence of a first exon, wherein the sequence of the first exon is immediately adjacent to an alternate splice junction, which is located between the first exon and a second exon. B, which is 3′ to A, is (a) at most 8 nucleotides in length, (b) comprises the sequence of a second exon, wherein the sequence of the second exon is immediately adjacent to the alternate splice junction, and (c) comprises at least 3 nucleotides that are unique to the second exon. The partner primer is an isolated oligonucleotide about 20 to about 25 nucleotides in length and comprises the sequence of, or the sequence that is complementary to, any sequence found either within about 600 nucleotides upstream of the alternate splice junction or within about 600 nucleotides downstream of the alternate splice junction. In a most preferred embodiment of the present invention, the targeted primer and partner primer are each 21 nucleotides in length and the partner primer comprises the sequence of, or the sequence that is complementary to, any sequence found either within 250 nucleotides upstream of the alternate splice junction or within 250 nucleotides downstream of the alternate splice junction.

The term “isoform” as used herein is defined as a variant or a related form of a protein that differs to some extent in amino acid sequence. According to Alberts et al., Molecular Biology of the Cell, 3^{rd} Ed., Garland Publishing, Inc., New York, 1994, “[isoforms] can be produced by different genes or by alternative splicing of RNA transcripts from the same gene.” In the instant case, two ER isoforms, α and β, are generated by different genes. However, additional isoforms of the ER exist as a result of alternatively splicing the α mRNA transcript and from alternatively splicing the β mRNA transcript. The term “alternative splicing” as used herein is defined by Matthews et al., Biochemistry, 2^{nd} Ed., The Benjamin/Cummings Publishing Co., Inc., Menlo Park, Calif. 1996, as “the splicing of a eukaryotic RNA transcript in different ways to include or exclude exons from the final mRNA.” The term “alternatively spliced exons” as used herein is defined as exons that have undergone alternative splicing. The term “alternate splice junction” as used herein is the point between two adjacent alternatively spliced exons that are positioned next to each other in an alternatively spliced mRNA transcript but not in a full-length, wild-type mRNA transcript. The term “full-length” as used herein pertains to a mRNA transcript comprising all of the exons of a gene. The term “wild-type” as used herein pertains to the lack of alternative splicing or mutations in a gene or a mRNA transcript. The terms “ER β₂, ER β₃, ER β₄, and ER β₅” as used herein pertain to mRNA transcripts comprising all of the exons of the ER β gene, except that the exon 8 in each of these differs from the “wild-type or ER β₁”.

In the above method as well as the other methods of the present invention, the targeted primer and the partner primer selectively hybridize to the cDNA template of the mRNA transcript of the ER under conditions that permit selective hybridization. Preferably, the hybridization is done under high stringency conditions. By “high stringency conditions,” it is meant that the primers specifically hybridize to target sequences of the cDNA template in an amount that is detectably stronger than non-specific hybridization. High stringency conditions, then, would be conditions, which would distinguish a polynucleotide with an exact complementary sequence of the cDNA template from one containing a only few small regions (e.g., 3-10 bases) with exact complementary sequence of the cDNA template. Such small regions of complementarity are more easily melted than a full-length complement of 14-17 or more bases and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the primer and the cDNA template of the mRNA transcript of the ER, and are particularly suitable for detecting expression of specific isoforms of the ER. It is generally appreciated that conditions can be rendered more stringent by the addition of increasing amounts of formamide.

Once hybridized, the complex comprising the targeted primer, partner primer, and cDNA template is contacted with one or more enzymes that facilitate template-dependent nucleic acid synthesis. Preferred enzymes include, for example, TaQMan DNA polymerase (Applied Biosystems, Foster City, Calif.). Multiple rounds of amplification, also referred to as “cycles,” are conducted until a sufficient amount of amplification product, or amplicons, is produced.

Various template-dependent processes are available to amplify the mRNA transcripts of the ER. A number of these processes are described in Sambrook et al., Molecular Cloning: A Laboratory Manual, 2^{nd} Ed., Cold Spring Harbor Press, Cold Spring Harbor, N.Y., 1989. One of the best-known amplification methods is the polymerase chain reaction (PCR). In a preferred embodiment of the invention, a real-time PCR is used when amplifying the mRNA transcripts of the ER. In real-time PCR, which is described in Bustin, J. Mol. Endocrinology 25: 169-193 (2000), PCRs are carried out in 96-well plates in the presence of a fluorogenic oligonucleotide probe that hybridizes to the amplicons. The fluorescent probes are double-labeled with a reporter fluorochrome and a quencher fluorochrome. When the probe anneals to the complementary sequence of the amplicon during PCR, the Taq polymerase, which possesses 5′ nuclease activity, cleaves the probe such that the quencher fluorochrome is displaced from the reporter fluorochrome, thereby allowing the latter to emit fluorescence. The resulting increase in emission, which is directly proportional to the level of amplicons, is monitored by a spectrophotometer. The cycle of amplification at which a particular level of fluorescence is detected by the spectrophotometer is called the threshold cycle, C_{T}. It is this value that is used to compare levels of amplicons.

In a preferred embodiment of the above inventive method and other methods of the present invention, wherein the method comprises amplifying a mRNA transcript of the β isoform of the ER, wherein the mRNA transcript comprises alternatively spliced exons, the targeted primer is selected from the group consisting of SEQ ID NO: 15, SEQ ID NO: 17, SEQ ID NO: 19, SEQ ID NO: 22, SEQ ID NO: 24, and SEQ ID NO: 25. In another preferred embodiment of these methods, the partner primer is selected from the group consisting of SEQ ID NO: 16, SEQ ID NO: 18, SEQ ID NO: 20, SEQ ID NO: 21, and SEQ ID NO: 23. In a more preferred embodiment of these methods, the pair of primers comprising a targeted primer and a partner primer is selected from the group consisting of SEQ ID NO: 15 and SEQ ID NO: 16; SEQ ID NO: 17 and SEQ ID NO: 18; SEQ ID NO: 19 and SEQ ID NO: 20; SEQ ID NO: 22 and SEQ ID NO: 21; SEQ ID NO: 24 and SEQ ID NO: 23; and SEQ ID NO: 25 and SEQ ID NO: 21.

The present invention further provides a method of generating an expression profile of mRNA transcripts of the β isoform of the ER, wherein said mRNA transcripts comprise alternatively spliced exons. The method comprises amplifying a mRNA transcript of the β isoform of the ER according to the above method, contacting the resulting amplicons with a detectably-labeled probe, detecting the detectably-labeled probe, and quantifying the detectably-labeled probe detected. By this method, the number of copies of a particular mRNA transcript of the β isoform of the ER can be determined.

In the above method of generating an expression profile of the β isoform of the ER, the amplification step can be repeated simultaneously or sequentially with a different pair of primers. In a preferred embodiment of the methods, this step is repeated such that a different pair of primers is used each time until all of the pairs of primers have been used.

Following amplification of the mRNA transcript, it can be desirable to separate the amplicons from the cDNA template and from the primers for the purpose of determining whether specific amplification has occurred. In one embodiment, the amplicons are separated by agarose, agarose-acrylamide or polyacrylamide gel electrophoresis, for example, using standard methods. See Sambrook et al. (1989), supra.

Alternatively, chromatographic techniques can be employed to effect separation. There are many kinds of chromatography which can be used in the context of the present inventive methods, e.g., adsorption, partition, ion-exchange and molecular sieve, and many specialized techniques for using them including column, paper, thin-layer and gas chromatography (Freifelder, Physical Biochemistry Applications to Biochemistry and Molecular Biology, 2^{nd} Ed., Wm. Freeman and Co., New York, N.Y. (1982)).

The amplicons must be visualized in order to confirm amplification of the mRNA of the ER. One typical visualization method involves staining of a gel with ethidium bromide and visualization under UV light. Alternatively, if the amplification products are integrally labeled with radio- or fluorometrically-labeled nucleotides, the amplification products then can be exposed to x-ray film or visualized under the appropriate stimulating spectra, following separation. In a preferred embodiment of the present inventive method, a labeled, nucleic acid probe can be brought into contact with the amplicons. The probe preferably is conjugated to a chromophore but may instead be radiolabeled or conjugated to a binding partner, such as an antibody or biotin, where the other member of the binding pair carries a detectable moiety (i.e., a label).

In a preferred embodiment of the above method as well as other methods of the present invention, wherein the method comprises contacting the amplicons with a detectably-labeled probe, the probe is an isolated oligonucleotide about 25 to about 30 nucleotides in length and comprises the sequence of, or the sequence complementary to, any sequence that is found between the sequence to which the targeted primer hybridizes and the sequence to which the partner primer hybridizes. The probe is preferably selected from the group consisting of SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 and SEQ ID NO 42. SEQ ID NO: 32 is the probe used with the pair of primers SEQ ID NO: 1 and SEQ ID NO: 2, SEQ ID NO: 33 is the probe used with the pair of primers SEQ ID NO: 3 and SEQ ID NO: 4, SEQ ID NO: 5 and SEQ ID NO: 4, SEQ ID NO: 8 and SEQ ID NO: 9, or SEQ ID NO: 8 and SEQ ID NO: 14, SEQ ID NO: 34 is the probe used with the pair of primers SEQ ID NO: 6 and SEQ ID NO: 7, or SEQ ID NO: 12 and SEQ ID NO: 13, SEQ ID NO: 35 is the probe used with the pair of primers SEQ ID NO: 10 and SEQ ID NO: 11, SEQ ID NO: 36 is the probe used with the pair of primers SEQ ID NO: 15 and SEQ ID NO: 16, SEQ ID NO: 37 is the probe used with the pair of primers SEQ ID NO: 17 and SEQ ID NO: 18, SEQ ID NO: 21 and SEQ ID NO: 22, or SEQ ID NO: 21 and SEQ ID NO: 25. SEQ ID NO: 38 is the probe used with the primer pairs SEQ ID NO: 19 and SEQ ID NO: 20 or SEQ ID NO: 23 and SEQ ID NO: 24; SEQ ID NO: 39 is the probe used with the pairs of primers SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, or SEQ ID NO: 26 and SEQ ID NO: 31; and SEQ ID NO: 42 is the probe used with the pair of primers SEQ ID NO: 40 and SEQ ID NO: 41.

In a more preferred embodiment of these present inventive methods, the probe comprises a fluorochrome at the 5′ end of the probe and a quencher fluorochrome at the 3′ end of the probe. It is most preferred that the fluorochrome is carboxy fluorescein (FAM) and the quencher fluorochrome is 6-carboxy-tetramethyl-rhodamine (TAMRA).

The present invention further provides a method of amplifying a mRNA transcript of the α isoform of the ER, wherein said mRNA transcript comprises alternatively spliced exons. The method comprises reverse-transcribing the mRNA transcript, amplifying the resulting cDNA using a pair of primers comprising a targeted primer and a partner primer. The pair of primers is selected from the group consisting of SEQ ID NO: 1 and SEQ ID NO: 2; SEQ ID NO: 3 and SEQ ID NO: 4; SEQ ID NO: 5 and SEQ ID NO: 4; SEQ ID NO: 6 and SEQ ID NO: 7; SEQ ID NO: 8 and SEQ ID NO: 9; SEQ ID NO: 10 and SEQ ID NO: 11; and SEQ ID NO: 12 and SEQ ID NO: 13.

A method of generating an expression profile of mRNA transcripts of the a isoform of the ER, wherein said mRNA transcripts comprise alternatively spliced exons is further provided by the present invention. The method comprises amplifying a mRNA transcript of the α isoform of the ER according to the above method, contacting the resulting amplicons with a detectably-labeled probe, detecting the detectably-labeled probe, and quantifying the detectably-labeled probe detected. By this method, the number of copies of a particular mRNA transcript of the α isoform of the ER can be determined.

The amplification step of the method of generating an expression profile of mRNA transcripts of the α isoform of the ER can be repeated simultaneously or sequentially with a different pair of primers, and it is preferable to repeat this step until all of the pairs of primers from the group have been used.

The method of generating an expression profile of mRNA transcripts of the α isoform can further comprise generating an expression profile of mRNA transcripts of the β isoform of the ER, wherein said mRNA transcripts comprise alternatively spliced exons. The method comprises amplifying a mRNA transcript of the β isoform of the ER in accordance with the method of amplifying a mRNA transcript of the β isoform of the ER of the present invention, contacting the resulting amplicons with a detectably-labeled probe, detecting the detectably-labeled probe, and quantifying the detectably-labeled probe detected. By this method, the number of copies of a particular mRNA transcript of the β isoform of the ER can be determined, in addition to the number of copies of a particular mRNA transcript of the α isoform of the ER.

In a preferred embodiment of the above method, the amplification step is repeated simultaneously or sequentially with a different pair of primers and, most preferably, it is repeated until all of the pairs of primers have been used.

The present invention further provides a method of generating an expression profile of the mRNA transcript of the full-length, wild-type α or β isoform of the ER. The method comprises reverse-transcribing the mRNA transcript and amplifying the resulting cDNA using a pair of primers comprising SEQ ID NO: 8 and SEQ ID NO: 14 (for the α isoform) or SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, or SEQ ID NO: 26 and SEQ ID NO: 31 (for the β isoform). The method further comprises contacting the resulting amplicons with a detectably-labeled probe, detecting the detectably-labeled probe, and quantifying the detectably-labeled probe detected. By this method, the number of copies of the full-length, wild-type mRNA transcript of the α or β isoform of the ER can be determined. This method can be carried out alone or in combination with the other methods of generating an expression profile described herein. In a preferred embodiment of present inventive methods of generating an expression profile of a mRNA transcript of full-length, wild-type α or β isoform of the ER, the amplification step is repeated simultaneously or sequentially with a different pair of primes and, most preferably, it is repeated until all of the pairs of primers have been used.

A method of generating the expression profile of a mRNA transcript of glyceraldehydes 3-phosphate dehydrogenase (GAPDH) is also provided by the present invention for the purpose of being used in conjunction with the methods of generating an expression profile described herein. This method comprises reverse-transcribing said mRNA transcript, amplifying the resulting cDNA using a pair of primers comprising SEQ ID NO: 40 and SEQ ID NO: 41, contacting the resulting amplicons with a detectably-labeled probe, detecting the detectably-labeled probe, and quantifying the detectably-labeled probe detected, upon which the number of copies of mRNA transcript of the GAPDH is determined. The number of copies of GAPDH mRNA transcripts serves as an internal control to which the number of copies of the mRNA trancript of the α or β isoform of the ER is compared.

In any of the above methods of generating an expression profile, a standard curve correlating a number of copies of in vitro-transcribed cRNA with a quantity of a detectably-labeled probe detected can be used to determine the number of copies of a particular mRNA transcript of the α or β isoform of the ER. The term “cRNA” as used herein encompasses RNA molecules that are complementary to DNA sequences, wherein the DNA sequences usually comprise coding regions of genes. In a preferred embodiment of the invention, a quantity of detectably-labeled probe detected is represented as the threshold cycle, C_{T}, which is the number of cycles required to achieve a particular level of fluorescence. In another preferred embodiment of the invention, the standard curve is generated by a method which is provided by the present invention and which comprises (i) in vitro transcribing RNA from a DNA plasmid, wherein said DNA plasmid encodes GAPDH, the wild-type protein of the α or β ER, an alternatively-spliced isoform of the α or β ER, or a fragment of either of the foregoing, wherein, when the fragment is that of an alternatively spliced isoform of the α or β ER, the fragment comprises the alternative splice junction, (ii) reverse transcribing the resulting cRNA, (iii) amplifying the resulting cDNA using the pair of primers that are used to amplify the wild-type protein of the α or β ER, the alternatively-spliced isoform of the α or β ER, or a fragment of either of the foregoing encoded in the DNA plasmid of step (i) selected from the group consisting of SEQ ID NO: 40 and SEQ ID NO: 41, SEQ ID NO: 1 and SEQ ID NO: 2; SEQ ID NO: 3 and SEQ ID NO: 4; SEQ ID NO: 5 and SEQ ID NO: 4; SEQ ID NO: 6 and SEQ ID NO: 7; SEQ ID NO: 8 and SEQ ID NO: 9; SEQ ID NO: 10 and SEQ ID NO: 11; SEQ ID NO: 12 and SEQ ID NO: 13; SEQ ID NO: 8 and SEQ ID NO: 14; SEQ ID NO: 15 and SEQ ID NO: 16; SEQ ID NO: 17and SEQ ID NO: 18; SEQ ID NO: 19and SEQ ID NO: 20; SEQ ID NO: 21 and SEQ ID NO: 22; SEQ ID NO: 23 and SEQ ID NO: 24; SEQ ID NO: 21 and SEQ ID NO: 25; SEQ ID NO: 26 and SEQ ID NO: 27; SEQ ID NO: 26 and SEQ ID NO: 28, SEQ ID NO: 26 and SEQ ID NO: 29, SEQ ID NO: 26 and SEQ ID NO: 30, and SEQ ID NO: 26 and SEQ ID NO: 31, (iv) contacting the resulting amplified products with a detectably-labeled probe, (v) detecting the detectably-labeled probe, (vi) quantifying the detectably-labeled probe detected, and (vii) repeating steps (ii)-(vi) at least 6 times, wherein the amount of cRNA used in step (ii) is different each time.

The present invention also provides a method of generating cRNA of a GAPDH or of an α or β isoform of the ER. The method comprises cloning a fragment of a coding sequence of the α or β isoform into a pCR®2.1-TOPO vector, wherein the fragment is at least about 500 base pairs in length, and then linearizing the resulting vector with the restriction enzyme BamHI. The method further comprises using T7 RNA polymerase to generate cRNA and then cleaving the vector with DNA-free™DNAse. Through this method, cRNA for the GAPDH or the α or β isoform of the ER is generated.

The present invention further provides a different method of generating cRNA in the case that the isoform is ERα5Δ. In this method, a fragment of a coding sequence of the ERα5Δ isoform is cloned into a pCR®2.1-TOPO vector. However, the restriction enzyme EcoRI is used to digest the resulting vector to obtain a second fragment containing the fragment of a ERα5Δ coding sequence. The resulting second fragment is subcloned into a pSG vector (Stratagene, La Jolla, Calif.), which is then digested with the restriction enzyme BglII. T7 RNA polymerase is used to generated cRNA, and the vector is cleaved with DNA-free™DNAse. The present inventive methods of generating cRNA can be employed in methods of generating a standard curve, which correlates a number of copies of in vitro-transcribed cRNA transcripts with a quantity of a detectably-labeled probe detected. The methods of generating cRNA described herein can be incorporated into methods of generating a standard curve, such as the one described above.

In any of the above methods, the source of mRNA transcripts can comprise any ER-expressing cells of a mammal, such as those cells obtained from the breast, ovary, brain, bone, female genital tract, male genital tract, gastrointestinal tract, nervous system, or immune system. The mRNA transcripts can be isolated from these cells according to standard methodologies, see, e.g., (Sambrook et al. (1989), supra). The term “mammal” as used herein encompasses any male or female mammal (e.g., human).

Further provided by the present invention is a method of determining breast cancer or a risk for breast cancer in a mammal. The method comprises comparing the expression profile of an ER-containing sample of the mammal with the expression profile of a control standard, wherein the control standard is a sample from a mammal, desirably of the same species, which is known to not have breast cancer. The expression profile can be obtained in accordance with any of the methods of generating an expression profile of the present invention. A difference in the expression profile of the mammal in comparison to the expression profile of the control standard is indicative of breast cancer or risk for breast cancer in the mammal. The difference could be, for example, an increase in the expression of ERα3Δ or ERα5Δ, which has been shown to be elevated in breast cancer tissues. The difference could also be an increase in the expression of ER isoforms that have multiple exons deleted, since multiple exon deletion isoforms of ERα were also found to increase in breast cancer tissues. Furthermore, the difference could be an increase in the expression of ERβ5Δ, which has been shown to be overexpressed in poorly differentiated, grade III tumors. Finally, the difference could be a reduction in ERβ5-6Δ expression, since decreased expression of this isoform was found in cancer tissues and not in matched normal tissues (Poola et al. (2002b), supra).

Also provided by the present invention is a method of prognosticating response of a mammal to a therapeutic treatment of breast cancer in the mammal. The method comprises comparing the expression profiles of ER-containing samples of the mammal obtained over the course of therapeutic treatment of the mammal. The expression profiles of this method can be obtained in accordance with any of the methods of generating an expression profile provided herein. In a preferred embodiment of the above method, the therapeutic treatment comprises any anti-estrogen therapy and in a more preferred embodiment, the anti-estrogen therapy is any estrogen-depriving drug, such as Tamoxifen and Femara.

A method of determining a hyperproliferative condition or a predisposition to a hyperproliferative condition in a mammal is also provided by the present invention. The method comprises comparing the expression profile of an ER-containing sample of the mammal with the expression profile of a control standard, wherein the control standard is a sample from a mammal, desirably of the same species, which is known to not have a hyperproliferative condition. The expression profiles of this method can be obtained in accordance with any of the methods of generating an expression profile provided herein. A difference in the expression profile of the mammal in comparison to the expression profile of the control standard is indicative of a hyperproliferative condition or a predisposition to a hyperproliferative condition in the mammal.

In a preferred embodiment of the present invention, the hyperproliferative condition is cancer, benign tumors, fibroids, polyps, cysts, and the like. In a more preferred embodiment, the cancer is cancer of the breast, ovary, uterus, bone, brain, female genital tract, male genital tract, gastrointestinal tract, nervous system, immune system, testis, or prostate.

Still further provided by the present invention is a method of screening candidate therapeutic treatments for an ER-sensitive condition. The method comprises comparing the expression profile of an ER-containing sample before treatment with the expression profile of the ER-containing sample after treatment, wherein said expression profiles have been obtained in accordance with the methods of generating an expression profile provided by the present invention.

The following examples further illustrate the invention but, of course, should not be construed as in any way limiting its scope.

### EXAMPLES

Abbreviations

For convenience, the following abbreviations are used herein: ER, estrogen receptor; ERα, α isoform of the ER; ERβ₁, full-length, wild-type β isoform of the ER; ERα2Δ, ERα isoform with exon 2 deleted; ERα3Δ, ERα isoform with exon 3 deleted; ERα2-3Δ, ERα isoform with exon 2 and exon 3 deleted; ERα4Δ, ER60 isoform with exon 4 deleted; ERα5Δ, ERα isoform with exon 5 deleted; ERα6Δ, ERα isoform with exon 6 deleted; ERα7Δ, ERα isoform with exon 7 deleted; ERβ₂, ERβ₃ ERβ₄ and ERβ₅, are the ERs that have distinct exon 8 sequences different from each other and from the wild-type; ERβ₁, or ERβ or ERβ wild-type are interchangeably used. ERβ2Δ, ERβ isoform with exon 2 deleted; ERβ3Δ, ERβ isoform with exon 3 deleted; ERβ4Δ, ERβ isoform with exon 4 deleted; ERβ5Δ, ERβ isoform with exon 5 deleted; ERβ6Δ, ERβ isoform with exon 6 deleted; ERβ5-6Δ, ERβ isoform with exon 5 and exon 6 deleted; PCR, polymerase chain reaction; CT, threshold cycle; RT-PCR, reverse transcription-polymerase chain reaction; GAPDH, glyceraldehyde 3-phosphate dehydrogenase; FAM, carboxy-fluorescein; TAMRA, 6-carboxy-tetramethyl-rhodamine; cRNA, complementary RNA; U, units; bp, basepair.

Materials

Omniscript reverse transcriptase kits were from QIAGEN Inc., Santa Clara, Calif. All of the primers used in the current study were synthesized by Gibco-BRL Life Technologies. TaqMan Universal PCR Master Mix (Cat # 4304437), RNAse inhibitor and random hexamers were purchased from Applied Biosystems (Foster City, Calif.). All of the 5′FAM and 3′TAMARA labeled oligonucleotide probes described here were synthesized at Applied Biosystems. MEGAscript™ kit and DNA-free™ DNAse were purchased from Ambion (Houston, Tex.). PCR quality water and Tris-EDTA buffer were from BioWhittaker (Walkersville, Md.). Total RNA from breast cancer cell lines and tumors were available from previous studies (Poola et al. Cancer 94: 615-623 (2002); Koduri et al., Cancer Res. Clin. Oncol 126: 219-297 (2000)). ERα wild-type and ERα 2Δ, ERα3Δ, ERα 2-3Δ, ERα4Δ, ERα5Δ, ERα6Δ, and ERα7Δ splice variant coding sequences, which were 1036 bp, 960 bp, 845 bp, 840 bp, 512 bp, 730 bp, 866 bp and 1000 bp in length, respectively, were cloned into pCR®2.1-TOPO plasmids, which were available from previous studies (Poola et al., J. Steroid. Biochem. Mol. Biol. 72: 249-258 (2000); ERβ1, ERβ2, ERβ4 and ERβ5 isoform coding sequences, 773 bp, 763 bp, 775 bp and 745 bp respectively, were amplified from human ovary cDNA using a sense primer in exon 4′CGGCAAGGCCAAGAGAAG3′ (position, exon 4, bp 824-841) and isoform specific anti-sense primers in exon 8. The anti-sense primers for ERβ1, ERβ2, ERβ4, and ERβ5 were 5′AGCACGTGGGCATTCAGC3′ (Position exon 8, bp 1580-1597 ), 5′GTCACTGCTCCATCGTTGCT3′ (position, exon 8, bp 1907-1887 (GenBank accession # AF051428), ′GTCTGGGTTTTATATCGTCTGCAA3′ (position, bp 294-271) (GenBank accession # AF061054) and 5′CACTTTTCCCAAATCACTTCACC3′ (position, bp 265-243) (GenBank accession # AF061055) respectively (Moore et al Biochem. Biophys. Res. Commun. 247, 75-78 (1998). These sequences were cloned into pCR®2.1-TOPO plasmid as described previously. ERβ exon 2Δ, 3Δ, 4Δ, 5Δ, 6Δ, and 5-6Δ splice variant coding sequences, 915 bp, 796 bp, 494 bp, 632 bp, 772 bp, and 633 bp respectively, were cloned into pCR®2.1-TOPO plasmids and were available from previous studies. (Poola et al., FEBS Letters 516: 133-138 (2002)). A 500-bp Glyceraldehyde 3-Phosphate dehydrogenase (GAPDH) coding sequence was amplified using an anti-sense primer, 5′ AAGGCTGAGAACGGGAAGCTTGTCATCAAT3′ (SEQ ID NO: 36) (Position, exon 3, bp 241-270), and a sense primer, 5′TTCCCGTCTAGCTCAGGGATGACCTTGCCC3′ (SEQ ID NO: 37)(Position, exon 7, bp 740-711), and the amplified product was cloned into the pCR®2.1-TOPO plasmid as described previously (Poola et al., J. Steroid. Biochem. Mol. Biol. 72: 249-258 (2000)).

### Example 1

This example demonstrates the preparation of cRNA using ERα isoform coding sequences cloned into pCR®2.1-TOPO plasmids as templates.

Absolute quantification of every ERα isoform transcript copy number was achieved by comparison with a standard graph constructed using known copy numbers of cDNA of that particular ERα isoform. Since reverse-transcribed tissue RNA is used for the quantification of test samples, the standard graph was also constructed with reverse-transcribed known copy numbers of cRNA. Complementary RNAs for eight ERα isoforms were generated by in vitro transcription using plasmids containing their respective nucleotide sequences as templates. The coding sequence of every isoform chosen for cRNA synthesis was 500 bp or higher, since the transcription using reagents from Ambion MEGAscript™ kit is optimal with templates larger than 500 bp. The cRNAs for every ERα isoform were generated in the following steps:

First, the pCR®2.1-TOPO vector containing the coding nucleotide sequences of wild-type or alternatively spliced ERα isoforms (5-10μg) were linearized by digesting with 20 units of BamHI in 25 μl for 4-6 hours. This enzyme linearizes pCR®2.1-TOPO vector 42 bp from the 5′ end of the cloned insert. The linearized plasmids were extracted with phenol/chloroform and precipitated. In the case of ERα5Δ, BamHI did not linearize the pCR®2.1-TOPO plasmid. Therefore, we adopted the following procedure: pCR®2.1-TOPO plasmid containing ERα5Δ sequence was digested with EcoRI to release the cloned sequences from two EcoRI sites that flank the insert. The released fragment was sub-cloned into pSG5 (Strategene, La Jolla, Calif.), and the cloned pSG5 was linearized by digesting with BglII.

Second, cRNA for every ERα isoform was generated by in vitro transcription of one to two μgs of linearized plasmids using T7 RNA polymerase and other reagents supplied in the Ambion MEGAscript™ kit and following the manufacturer's protocol. Briefly, the transcription reaction mixture (20 μl) contained 1 μg of linearized ERα isoform-pCR®2.1-TOPO, 2 μl of 10× buffer, 2 μl each of 75 mM NTPs, and 2 μl of T7 RNA polymerase. The reaction mixture was incubated at 37° C. for 2-4 hours. This generally produced about 20-50 μg of cRNA.

Third, since the cRNAs generated by this procedure also contained the template plasmids which interfere with absolute quantification of RNA transcripts, they were removed by digesting with DNA-free™ DNAse. cRNAs (20-25 μg) were incubated with 2-4 units of DNAse for 4 hours and the digested RNA was precipitated after extraction with phenol/chloroform. DNAse digestion was repeated to remove any trace amounts of plasmid DNAs. The cRNAs prepared by this procedure were found to be devoid of any plasmid DNAs by PCR without reverse transcription. Concentration of cRNAs was determined by measuring the optical density at 260 nm. The cRNAs for ERβ isoforms were also prepared by the above procedures.

### Example 2

This example demonstrates a procedure for reverse transcription and conventional PCR of RNAs from breast cancer cell lines and breast cancer tumors.

The in vitro-transcribed cRNAs from Example 1 and the total RNAs from breast cancer cell lines/tumors were reverse-transcribed using Omniscript reverse transcriptase as previously described (Poola et al., Cancer 94: 615-623 (1990); and Koduri et al., Steroids 66: 17-23 (2001)), but with a minor modification. Briefly, the cRNA or total RNA was denatured by heating for 3 minutes at 65° C., cooled on ice, and incubated with a reverse transcriptase reaction mixture, which contained 1 μg of total RNA, 10 U of RNAse inhibitor, 0.5 mM each of dNTPs, 1 μM random hexamers, and 4 U of Omniscript reverse transcriptase in a total volume of 20 μl. For reverse transcription, tubes were incubated at 42° C. for 60 min, followed by 95° C. for 5 min and then rapidly cooled. Conventional PCR reactions were performed in an automatic thermal cycler (MJ Research) as previously described (Poola et al., Anal. Biochem. 258: 209-215 (1998)) in a total volume of 12.5 μl containing the cDNA reverse-transcribed from 125 ng of total RNA, 1×PCR buffer, 1×Q solution, 200 μM each of dNTPs, 2 μM each of sense and anti-sense primers and 0.6 U of HotStartTaq polymerase. The PCR conditions were as follows: initial denaturation for 1 min at 95° C., followed by 94° C. for 1 min, annealing for 1 min at the specified temperature depending on the primer pair used, extension for 2 min at 72° C. for 40 cycles and final extension for 15 min at 72° C. The annealing temperature for amplification of ERα wild-type, ERα3Δ and ERα7Δ transcripts was 55° C. For amplication of ERα2Δ, ERα2-3Δ, ERα4Δ and ERα6Δ, the annealing temperature was 60° C., and for ERα5Δ, the temperature was 65° C. The ER products (3.0 μl) generated by conventional PCR procedures were separated by electrophoresis in 1% Nu Sieve agarose gels in Tris-Acetic Acid-EDTA buffer and detected by ethidium bromide staining. PCR products were purified by gel extraction, cloned into pCR®2.1-TOPO vector, and identified by sequence analysis as described previously (Poola et al., J. Steroid. Biochem. Mol. Biol. 72: 249-258 (2000)).

### Example 3

This example demonstrates a procedure for the absolute quantification of eight ERα isoform transcript copy numbers by Real-Time PCR.

Absolute quantification of ERα wild-type, ERα2Δ, ERα2-3Δ. ERα4Δ, and ERα5Δ transcript copy numbers was achieved by quantitative Real-time PCR in ABI Prism GeneAmp 7900HT Sequence Detection System at 50% Ramp rate. For the quantification of ERα3Δ, ERα6Δ, and ERα7Δ transcripts, Real-Time PCRs were conducted at 25% ramp rate. A typical Real-Time PCR reaction mixture contained cDNA prepared from reverse transcription of 0.5-5 nanograms of tumor tissue/cell line total RNA, 0.04 micromolar each of sense and anti-sense primers, 0.05 micromolar 5′FAM and 3′TAMARA labeled oligonucleotide probe and 1× Taqman Universal PCR Mix in a total volume of 25 μl. PCR conditions were initially held at 50° C. for 2 minutes, followed by denaturation for 10 minutes at 95° C., and denaturation for 15 seconds at 95° C. in the subsequent cycles and annealing and extension for 1 min at 60° C. for 40 cycles. An additional step of annealing at 55° C. for 15 s was added before the amplification step at 60° C. whenever the assay was conducted at a 25% ramp rate. A standard graph for every ERα isoform was simultaneously generated using 10², 10³, 10⁴, 10⁵, 10⁶, 10⁷, 10⁸, and 10⁹ copies of its reverse-transcribed cRNA. The tumor cDNAs, primers and probe were diluted on ice to the required concentration immediately prior to conducting PCRs. All the samples were amplified in triplicate and Real-Time PCRs were repeated four times for every isoform. GAPDH copy numbers were determined using its specific primer pair and probe in a given amount of cDNA of every tumor/cell line using the procedure described above.

### Example 4

This example demonstrates a procedure for the determination of various copy numbers of an ERα isoform.

For the determination of the copy number of the ERα6Δ isoform, the number of bases was first calculated to be 980 bp (866 bp from coding sequence and 114 bp from the vector (72 bp from T7 promoter site to downstream of the insert and 42 bp upstream of the insert to BamHI site in pCR®2.1-TOPO vector)). The molecular weight of the above in vitro-transcribed cRNA was obtained by multiplying the number of bases (980) with 660 (MW of one base pair) (980×660=6.46×10⁵ daltons). The number of moles per liter for a given concentration of cRNA (for example, 1 μg/20 μl or 5×10⁻² g/liter) was calculated by dividing it with its molecular weight (5×10⁻² g/liter over 6.46×10⁵=7.7×10⁻⁸ moles/liter). Since one mole of a compound contains 6.023×10²³ copies (Avagadro's number), the number of copies in a given number of moles was calculated by multiplying it with Avagadro's number (7.7×10⁻⁸×6.023×10²³ copies/liter=4.63×10¹⁶ copies/liter). A known amount of cRNA was reverse-transcribed and the number of copies/μl was calculated as above and diluted freshly on ice to the required number of copies just before conducting Real-Time PCRs.

### Example 5

This example demonstrates the specificity of the primers used to amplify the ER isoforms.

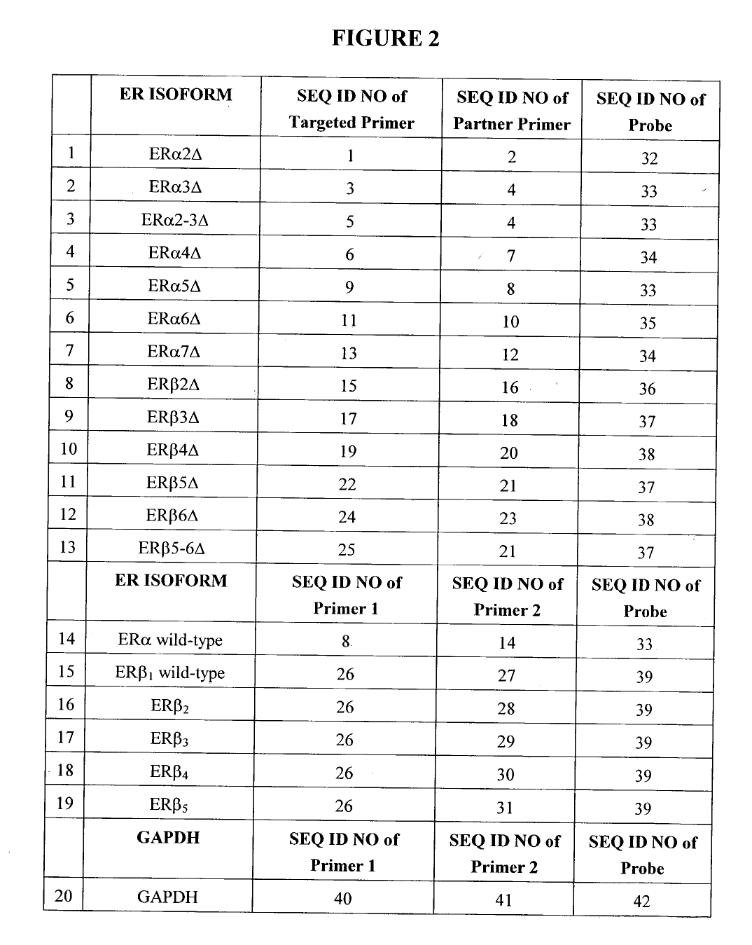

Conventional PCR in accordance with Example 3 was performed to determine the specificity of the primer pairs for wild-type ERα, ERα2Δ, ERα3Δ, ERα2-3Δ, ERα4Δ, ERα5Δ, ERα6Δ, and ERα7Δ that are disclosed in FIG. 2. As expected, the PCR products for wild-type ERα, ERα2Δ, ERα3Δ, ERα2-3Δ, ERα4Δ, ERα5Δ, ERα6Δ, and ERα7Δ isoforms were 248 bp, 162, bp, 217 bp, 217 bp, 231 bp, 232 bp, 196 bp, and 177 bp, respectively. The specificity of the primer pairs used in the PCR reactions was demonstrated by the fact that the lanes of the agarose gel containing samples that had PCR reaction mixtures with either of the template derived from the total RNA from MCF-7 cells or from a corresponding cRNA had a band of expected size, while lanes containing samples that had PCR reaction mixtures with no template did not have a band.

This example demonstrates that the primer pairs used to amplify the various isoforms of ERα were specific to that isoform.

### Example 6

This example demonstrates the sensitivity of the primers in Real Time PCR.

The sensitivity of the primers together with 5′FAM and 3′TAMARA labeled probes were tested using ERα isoform DNAs prepared by reverse transcription of various copy numbers of their respective cRNAs as described in Examples 1 and 2. Sensitivity was tested with copies ranging from 10² to 10⁹ of reverse-transcribed cRNA. The results obtained for eight isoforms are presented in Table 1a. The results obtained for ten ERβ isoforms are presented in Table 1b.

TABLE 1a
Sensitivity of Detection of Ten ERα Isoform Copy
Numbers by Real-Time PCR with Reverse-Transcribed cRNA

| | Sensitivity (copies detected) at | |
|---|---|---|
| Isoform | 50% Ramp Rate | 25% RampRate |
| Wild-type ERα | 10² | ND |
| ERα2Δ | 10² | ND |
| ERα3Δ | 10⁴ | 10² |
| ERα2-3Δ | 10² | ND |
| ERα4Δ | 10² | ND |
| ERα5Δ | 10² | ND |
| ERα6Δ | 10³ | 10² |
| ERα7Δ | 10³ | 10² |

ND. Not Determined.

TABLE 1b
Sensitivity of Detection of Ten ERβ Isoform Copy
Numbers by Real-Time PCR with Reverse-Transcribed cRNA

| | Sensitivity (copies detected) at | |
|---|---|---|
| Isoform | 50% Ramp Rate | 25% Ramp Rate |
| ERβ1 (Wild | 10² | ND |
| Type) | | |
| ERβ2 | 102 | ND |
| ERβ4 | 103 | 102 |
| ERβ5 | 102 | ND |
| ERβ Exon 2Δ | 102 | ND |
| ERβ Exon 3Δ | 103 | 102 |
| ERβ Exon 4Δ | 102 | ND |
| ERβ Exon 5Δ | 103 | 103 |
| ERβ Exon 6Δ | 103 | 102 |
| ERβ Exon 5-6Δ | 103 | 102 |

ND. Not Determined.

At a 50% ramp rate, the primer pairs and probes for wild-type, ERα2Δ, ERα2-3Δ, ERα4Δ, and ERα5Δ detected as low as 10² copies. At the 50% ramp rate, the primer pairs and probes of ERα3Δ, ERα6Δ and ERα7Δ detected 10⁴, 10³, and 10³ copies, respectively. To improve the sensitivity of detection, these primer pairs and probes were tested at a 25% ramp rate. At this ramp rate, the above three detected 10² copies. Thus, it appears that the sensitivity of detection depends on the ramp rate at which the Real-Time PCR is performed.

Next, the above ERα primer pairs and probes were tested for their sensitivity to detect cDNAs prepared from two breast cancer tissues. For this, the tumor cDNAs were reverse-transcribed from 50 ng of total RNA in accordance to Example 2 and were serially double diluted and amplified by Real-Time PCR in accordance with Example 3. The sensitivity is defined as the dilution (minimum amount of reverse-transcribed total RNA) that gives the detectable amplification plot. The results obtained with two tumors for the isoforms are presented in Table 2a. Sensitivity of detection of ten ERβ isoforms were tested for their sensitivity to detect cDNAs prepared from normal breast, ovary and uterus and the results are presented in Table 2b.

TABLE 2a
Sensitivity of Dectection of Eight
ERα Isoforms in Tumors by Real-Time PCR

| | Sensitivity of Detection (pg of | |
|---|---|---|
| | reverse-transcribed total RNA) | |
| ERα Isoform | Tumor I | Tumor II |
| Wild-type | 97 | 48 |
| ERα | | |
| ERα2Δ | 195 | 195 |
| ERα3Δ | 780 | 390 |
| ERα2-3Δ | 780 | 390 |
| ERα4Δ | 1560 | 780 |
| ERα5Δ | 390 | 390 |
| ERα6Δ | 12500 | 12500 |
| ERα7Δ | 195 | 97 |

TABLE 2b
Sensitivity of Detection of ERβ Isoforms
in Human Breast, Ovary and Uterus by Real-Time PCR

| | Sensitivity of Detection | | |
|---|---|---|---|
| | (ng of reverse transcribed total RNA) | | |
| Isoform | Breast | Ovary | Uterus |
| ERβ1 | 1.56 | 0.0975 | 12.5 |
| ERβ2 | 3.125 | 0.195 | 3.125 |
| ERβ4 | 6.25 | 0.78 | ND |
| ERβ5 | 0.39 | 0.0975 | 1.56 |
| ERβ Exon 2Δ | 100 | 1 | ND |
| ERβ Exon 3Δ | ND | 100 | ND |
| ERβ Exon 4Δ | 10 | 5 | 5 |
| ERβ Exon 5Δ | 5 | 1 | 1 |
| ERβ Exon 6Δ | ND | 1 | ND |
| ERβ Exon 5-6Δ | 1 | 1 | 1 |

ND. Not Detected, ERβ3 not detected in any of the above tissues.

As seen in Table 2a, the sensitivity of detection varied among isoforms and among the two tumors. These results suggest that the wild-type isoform could be detected with cDNA prepared from reverse transcription of as little as 50-100 pg of total RNA from tumors. Among the splice variants, ERα7Δ and ERα2Δ were detected with very low amounts of reverse-transcribed total RNA (100-200 pg). The sensitivity of detection of other variants ranged from 400-800 pg, except for ERα6Δ. The minimum amount of total RNA required to detect this variant was about 12 ng. The ten ERβ were expressed at different levels in different tissues.

This example demonstrates that the method described here is over one thousand-fold more sensitive compared to the template competition method, which requires ˜10 μg of total RNA for profiling eight ERα isoforms. The high sensitivity of the method will be particularly suitable for profiling ERα isoforms in samples from stereotactic biopsies, ductal lavage or even nipple aspirate fluids.

### Example 7

This example demonstrates the practice of the procedures of Examples 3 and 4 for the absolute quantification of eight ERα isoforms and ten ERβ isoforms.

cDNAs from six breast cancer cell lines and ten breast cancer tissues were subjected to Real-Time PCRs. For the quantification of wild-type receptor, cDNAs were generally diluted such that the amount of reverse-transcribed total RNA used was 500 pg to 1 ng. For the quantification of all splice variants except ERα6Δ, the reverse-transcribed total RNA ranged from 1-5 ng. The ERα6Δ transcript was amplified using 15-20 ng of reverse-transcribed total RNA. The number of copies of every isoform in a given amount of sample was determined in comparison with a standard graph constructed with known copy numbers (10²-10⁹copies) of its respective reverse-transcribed cRNA as described in Example 2. A standard graph was constructed each time a quantitation of the test sample was performed. The housekeeping gene, GAPDH, was quantified as an internal standard. The number of copies of every isoform was normalized to 10¹⁰ copies of GAPDH. The mRNA copy numbers of eight ERα isoforms from six breast cancer cell lines and ten breast cancer tissues are presented in Table 3a and ten ERβ isoforms in ovary, breast, uterus and bone tissues are presented in Table 3b.

TABLE 3a
ERα Isoform Copies per 10¹⁰ Copies of GAPDH in Breast Cancer Cell Lines and Tumors
by Real-Time PCR

| Cell or | | | | | | | | |
|---|---|---|---|---|---|---|---|---|
| Tumor | ERαWT | ERα2Δ | ERα3Δ | ERα2-3Δ | ERα4Δ | ERα5Δ | ERα6Δ | ERα7Δ |
| MCF-7 | 2.4 × 10⁷ | 4.3 × 10⁵ | 2.1 × 10⁵ | 1.5 × 10⁵ | 2.5 × 10⁵ | 1.3 × 10⁶ | 3.6 × 10⁴ | 5.1 × 10⁶ |
| T47D | 3.3 × 10⁷ | 2.7 × 10⁵ | 1.2 × 10⁵ | 1.6 × 10⁵ | 3.1 × 10⁵ | 1.2 × 10⁶ | 3.6 × 10⁴ | 4.6 × 10⁶ |
| ZR-75 | 2.5 × 10⁶ | 1.7 × 10⁵ | 6.8 × 10⁴ | 6.2 × 10⁴ | 6.2 × 10⁴ | 1.4 × 10⁵ | 8.6 × 10³ | 1.5 × 10⁶ |
| LCC1 | 3.6 × 10⁷ | 7.1 × 10⁵ | 8.3 × 10⁵ | 2.9 × 10⁵ | 3.6 × 10⁵ | 2.3 × 10⁶ | 2.4 × 10⁴ | 1.1 × 10⁷ |
| LCC2 | 4.7 × 10⁷ | 1.5 × 10⁶ | 7.7 × 10⁵ | 4.4 × 10⁵ | 5.6 × 10⁵ | 4.7 × 10⁶ | 2.3 × 10⁴ | 1.6 × 10⁷ |
| LCC9 | 1.7 × 10⁷ | 8.0 × 10⁵ | 2.7 × 10⁵ | 2.5 × 10⁵ | 1.2 × 10⁵ | 6.4 × 10⁵ | 3.7 × 10³ | 8.7 × 10⁶ |
| Tumor 1 | 4.2 × 10⁷ | 8.0 × 10⁶ | 6.4 × 10⁵ | 1.5 × 10⁶ | 8.2 × 10⁵ | 1.4 × 10⁶ | 0 | 5.0 × 10⁶ |
| Tumor 2 | 2.3 × 10⁷ | 1.7 × 10⁶ | 6.3 × 10⁵ | 9.3 × 10⁵ | 1.6 × 10⁵ | 8.0 × 10⁵ | 6.5 × 10⁴ | 1.7 × 10⁶ |
| Tumor 3 | 3.6 × 10⁶ | 8.3 × 10⁵ | 0 | 2.6 × 10⁵ | 9.9 × 10⁵ | 1.1 × 10⁵ | 0 | 1.8 × 10⁶ |
| Tumor 4 | 2.0 × 10⁸ | 3.5 × 10⁶ | 1.6 × 10⁶ | 3.4 × 10⁶ | 6.4 × 10⁵ | 3.9 × 10⁶ | 0 | 8.0 × 10⁶ |
| Tumor 5 | 4.4 × 10⁷ | 1.3 × 10⁶ | 1.4 × 10⁵ | 2.7 × 10⁵ | 1.5 × 10⁵ | 6.5 × 10⁵ | 6.1 × 10³ | 5.6 × 10⁵ |
| Tumor 6 | 1.1 × 10⁶ | 4.5 × 10⁶ | 2.8 × 10⁴ | 4.7 × 10⁴ | 1.7 × 10⁴ | 4.7 × 10⁴ | 0 | 3.6 × 10⁶ |
| Tumor 7 | 2.4 × 10⁷ | 3.8 × 10⁶ | 2.0 × 10⁵ | 3.3 × 10⁵ | 8.2 × 10⁴ | 1.5 × 10⁵ | 0 | 1.28 × 10⁷ |
| Tumor 8 | 4.7 × 10⁷ | 5.9 × 10⁶ | 1.5 × 10⁶ | 1.1 × 10⁶ | 5.7 × 10⁵ | 1.7 × 10⁶ | 1.3 × 10⁴ | 4.6 × 10⁶ |
| Tumor 9 | 4.0 × 10⁷ | 2.0 × 10⁶ | 1.5 × 10⁵ | 9.5 × 10⁵ | 2.3 × 10⁵ | 6.8 × 10⁵ | 3.2 × 10³ | 1.8 × 10⁶ |
| Tumor 10 | 6.5 × 10⁶ | 3.3 × 10⁵ | 0 | 6.8 × 10⁶ | 1.3 × 10⁵ | 2.2 × 10⁵ | 0 | |

TABLE 3b
Ten ERβ Isoform Copies per 10¹⁰ of GAPDH in Breast, Ovary, Uterus and Bone Tissues
by Real-Time PCR

| | ERβ1 | | | | ERβ | ERβ | ERβ | ERβ | ERβ | ERβ |
|---|---|---|---|---|---|---|---|---|---|---|
| Cell/ | Wild | | | | exon | exon | exon | exon | exon | exon |
| Tissue | Type | ERβ2 | ERβ4 | ERβ5 | 2Δ | 3Δ | 4Δ | 5Δ | 6Δ | 5-6Δ |
| Breast | 5.8 × 10⁵ | 6.2 × 10⁴ | 3.3 × 10⁵ | 7 × 10⁶ | 3.3 × 10³ | 0 | 1.1 × 10⁶ | 2.3 × 10⁶ | 4.0 × 10⁴ | 6.2 × 10⁶ |
| Ovary | 3.6 × 10⁷ | 1.4 × 10⁷ | 1.2 × 10⁶ | 8 × 10⁷ | 9.6 × 10⁶ | 5 × 10⁵ | 2.0 × 10⁶ | 2 × 10⁷ | 3.7 × 10⁶ | 7.9 × 10⁶ |
| Uterus | 1.4 × 10⁴ | 0 | 0 | 3.2 × 10⁷ | 3.5 × 10⁴ | 0 | 1.2 × 10⁶ | 1.5 × 10⁷ | 3.5 × 10⁴ | 8.5 × 10⁷ |
| Bone | 0 | 1.1 × 10³ | 0 | 2.7 × 10⁵ | 0 | 0 | 0 | 0 | 0 | 7.6 × 10³ |
| MCF-7 | 8.5 × 10⁴ | 1.1 × 10⁵ | 0 | 2.8 × 10⁵ | 4.2 × 10³ | 0 | 3.5 × 10⁴ | 5.7 × 10⁵ | 0 | 5.7 × 10⁴ |
| MDA- | 1.2 × 10⁴ | 1.8 × 10⁴ | 0 | 2 × 10⁵ | 2.0 × 10³ | 0 | 0 | 6.0 × 10⁴ | 0 | 1.5 × 10⁴ |
| MB- | | | | | | | | | | |
| 231 | | | | | | | | | | |
| ERα⁺ | 9.6 × 10⁴ | 1.68 × 10⁴ | 0 | 2.4 × 10⁶ | 6.4 × 10⁵ | 0 | 0 | 1.4 × 10⁶ | 0 | 5.0 × 10⁶ |
| Breast | | | | | | | | | | |
| Tumor | | | | | | | | | | |
| ERα⁻ | 1.8 × 10⁵ | 8.0 × 10⁴ | 0 | 2 × 10⁵ | 1.4 × 10⁴ | 0 | 1.3 × 10⁴ | 1.8 × 10⁶ | 3.5 × 10⁴ | 6.0 × 10⁴ |
| Breast | | | | | | | | | | |
| Tumor | | | | | | | | | | |

In all of the tumors and cell lines tested, the wild-type α receptor transcripts are the most abundant, followed by ERα7Δ and ERα2Δ (Poola, Analytical Biochemistry 2003 (In Press)). The ERα6Δ transcript appears to be the least abundant as reported previously (Poola et al., J. Steroid. Biochem. Mol. Biol 72: 249-258 (2000)). Two tumor tissues did not express any ERα3Δ. The values presented in Table 3 show that both tumors and cell lines express significant amounts of splice variants. The strategies used here for the quantification of ERα splice variants can be applied to splice variants of other genes. Among four tissues tested, ovary expresses higher ERβ copy numbers than any other tissue. Bone tissue seems to express ERβ2 and ERβ5, although it does not express ERβ1.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. An isolated oligonucleotide consisting essentially of the nucleotide sequence selected from the group consisting of SEQ ID NO: 8, SEQ ID NOS: 15-27, SEQ ID NO: 40, and SEQ ID NO: 41.

2. An isolated oligonucleotide consisting essentially of the nucleotide sequence selected from the group consisting of SEQ ID NOS: 32-39 and SEQ ID NO: 42.

3. A method of amplifying a mRNA transcript of the β isoform of the estrogen receptor (ER), wherein said mRNA transcript comprises alternatively spliced exons, which method comprises:
(i) reverse transcribing said mRNA transcript,
(ii) amplifying the resulting cDNA using a pair of primers comprising a targeted primer and a partner primer,
wherein the targeted primer is an isolated oligonucleotide comprising two parts, A and B,
wherein A is about 16 to about 25 nucleotides in length and comprises the sequence of a first exon,
wherein the sequence of the first exon is immediately adjacent to an alternate splice junction,
wherein B is (a) at most 8 nucleotides in length, (b) comprises the sequence of a second exon, wherein the sequence of the second exon is immediately adjacent to the alternate splice junction, and (c) comprises at least 3 nucleotides that are unique to the second exon,
wherein the alternate splice junction is between the first exon and the second exon and A is 5′ to B, and
wherein the partner primer is an isolated oligonucleotide about 20 to about 25 nucleotides in length and comprises the sequence of or the sequence that is complementary to any sequence found either within about 600 nucleotides upstream of the alternate splice junction or within about 600 nucleotides downstream of the alternate splice junction, whereupon a mRNA transcript of the β isoform of the ER is amplified.

4. The method of claim 3, wherein the targeted primer is selected from the group consisting of:
SEQ ID NO: 15,
SEQ ID. NO: 17,
SEQ ID NO: 19,
SEQ ID NO: 22,
SEQ ID NO: 24, and
SEQ ID NO: 25,
wherein the partner primer is selected from the group consisting of:
SEQ ID NO: 16,
SEQ ID NO: 18,
SEQ ID NO: 20,
SEQ ID NO: 21, and
SEQ ID NO: 23, or
wherein the pair of primers comprises a targeted primer and a partner primer selected from the group consisting of:
SEQ ID NO: 15 and SEQ ID NO: 16,
SEQ ID NO: 17 and SEQ ID NO: 18,
SEQ ID NO: 19 and SEQ ID NO: 20,
SEQ ID NO: 22 and SEQ ID NO: 21,
SEQ ID NO: 24 and SEQ ID NO: 23, and
SEQ ID NO: 25 and SEQ ID NO: 21.

5. A method of generating an expression profile of mRNA transcripts of the β isoform of the ER, wherein said mRNA transcripts comprise alternatively spliced exons, which method comprises:
(i) amplifying a mRNA transcript of the β isoform of the ER according to the method of claim 3,
(ii) contacting the resulting amplicons with a detectably-labeled probe,
(iii) detecting the detectably-labeled probe, and
(iv) quantifying the detectably-labeled probe detected,
whereupon the number of copies of a particular mRNA transcript of the β isoform of the ER is determined.

6. The method of claim 5, which further comprises generating the expression profile of the mRNA transcripts of the full-length, wild-type α or β isoform of the ER, which method comprises:
(i) reverse transcribing said mRNA transcript,
(ii) amplifying the resulting cDNA using a pair of primers comprising SEQ ID NO: 8 and SEQ ID NO: 14 or SEQ ID NO: 26 and SEQ ID NO: 27, or SEQ ID NO: 26 and SEQ ID NO: 28 or SEQ ID NO: 26 and SEQ ID NO: 29 or SEQ ID NO: 26 and SEQ ID NO: 30 or SEQ ID NO: 26 and SEQ ID NO: 31
(iii) contacting the resulting amplicons with a detectably-labeled probe,
(iv) detecting the detectably-labeled probe, and
(v) quantifying the detectably-labeled probe detected,
whereupon the number of copies of the full-length, wild-type mRNA transcript of the α or β isoform of the ER is determined.

7. The method of claim 5, wherein the targeted primer is selected from the group consisting of:
SEQ ID NO: 15,
SEQ ID NO: 17,
SEQ ID NO: 19,
SEQ ID NO: 22,
SEQ ID NO: 24, and
SEQ ID NO: 25,
wherein the partner primer is selected from the group consisting of:
SEQ ID NO: 16,
SEQ ID NO: 18,
SEQ ID NO: 20,
SEQ ID NO: 21, and
SEQ ID NO: 23, or
wherein the pair of primers comprises a targeted primer and a partner primer selected from the group consisting of:
SEQ ID NO: 15 and SEQ ID NO: 16,
SEQ ID NO: 17 and SEQ ID NO: 18,
SEQ ID NO: 19 and SEQ ID NO: 20,
SEQ ID NO: 22 and SEQ ID NO: 21,
SEQ ID NO: 24 and SEQ ID NO: 23, and
SEQ ID NO: 25 and SEQ ID NO: 21.

8. The method of claim 5, wherein the probe is an isolated oligonucleotide about 25 to about 30 nucleotides in length and comprises the sequence of or the sequence that is complementary to any sequence that is found between the sequence to which the targeted primer hybridizes and the sequence to which the partner primer hybridizes, wherein the probe optionally comprises a fluorochrome at the 5′ end of the probe and a quencher fluorochrome at the 3′ end of the probe.

9. The method of claim 8, wherein the probe is selected from the group consisting of:
SEQ ID NO: 33,
SEQ ID NO: 36,
SEQ ID NO: 37,
SEQ ID NO:38,
SEQ ID NO: 39 and
SEQ ID NO: 42,
wherein SEQ ID NO: 33 is the probe used with the pair of primers SEQ ID NO: 8 and SEQ ID NO: 14,
SEQ ID NO: 36 is the probe used with the pair of primers SEQ ID NO: 15 and SEQ ID NO: 16,
SEQ ID NO: 37 is the probe used with the pair of primers SEQ ID NO: 17 and SEQ ID NO: 18, SEQ ID NO: 21 and SEQ ID NO: 22, or SEQ ID NO: 21 and SEQ ID NO: 25,
SEQ ID NO: 38 is the probe used with the pair of primers SEQ ID NO: 19 and SEQ ID NO: 20, or SEQ ID NO: 23 and SEQ ID NO: 24,
SEQ ID NO: 39 is the probe used with the pair of primers SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, or SEQ ID NO: 26 and SEQ ID NO: 31 and
SEQ ID NO: 42 is the probe used with the pair of primers SEQ ID NO: 40 and SEQ ID NO: 41.

10. The method of claim 5, wherein a standard curve correlating a number of copies of in vitro-transcribed cRNA with a quantity of a detectably-labeled probe detected is used to determine the number of copies of a particular mRNA transcript of the α or β isoform of the ER.

11. The method of claim 5, which further comprises generating the expression profile of a mRNA transcript of Glyceraldehyde 3-Phosphate Dehydrogenase (GAPDH), which method comprises:
(i) reverse transcribing said mRNA transcript,
(ii) amplifying the resulting cDNA using a pair of primers comprising SEQ ID NO: 40 and SEQ ID NO: 41,
(iii) contacting the resulting amplicons with a detectably-labeled probe,
(iv) detecting the detectably-labeled probe, and
(v) quantifying the detectably-labeled probe detected,
whereupon the number of copies of mRNA transcript of the GAPDH is determined.

12. The method of claim 11, wherein the probe is an isolated oligonucleotide about 25 to about 30 nucleotides in length and comprises the sequence of or the sequence that is complementary to any sequence that is found between the sequence to which the targeted primer hybridizes and the sequence to which the partner primer hybridizes, wherein the probe optionally comprises a fluorochrome at the 5′ end of the probe and a quencher fluorochrome at the 3′ end of the probe.

13. The method of claim 12, wherein the probe is selected from the group consisting of:
SEQ ID NO: 33,
SEQ ID NO: 36,
SEQ ID NO: 37,
SEQ ID NO: 38,
SEQ ID NO: 39 and
SEQ ID NO: 42,
wherein SEQ ID NO: 33 is the probe used with the pair of primers SEQ ID NO: 8 and SEQ ID NO: 14,
SEQ ID NO: 36 is the probe used with the pair of primers SEQ ID NO: 15 and SEQ ID NO: 16,
SEQ ID NO: 37 is the probe used with the pair of primers SEQ ID NO: 17 and SEQ ID NO: 18, SEQ ID NO: 21 and SEQ ID NO: 22, SEQ ID NO: 21 and SEQ ID NO: 25,
SEQ ID NO: 38 is the probe used with the pair of primers SEQ ID NO: 19 and SEQ ID NO: 20, or SEQ ID NO: 23 and SEQ ID NO: 24,
SEQ ID NO: 39 is the probe used with the pair of primers SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, or SEQ ID NO: 26 and SEQ ID NO: 31, and
SEQ ID NO: 42 is the probe used with the pair of primers SEQ ID NO: 40 and SEQ ID NO: 41.

14. The method of claim 11, wherein a standard curve correlating a umber of copies of in vitro-transcribed cRNA with a quantity of a detectably-labeled probe detected is used to determine the number of copies of a particular mRNA transcript of the α or β isoform of the ER or the number of copies of the mRNA transcript of the GAPDH.

15. A method of amplifying a mRNA transcript of the α isoform of the ER, wherein said mRNA transcript comprises alternatively spliced exons, which method comprises:
(i) reverse transcribing said mRNA transcript,
(ii) amplifying the resulting cDNA using a pair of primers comprising a targeted primer and a partner primer selected from the group consisting of:
SEQ ID NO: 1 and SEQ ID NO: 2,
SEQ ID NO: 3 and SEQ ID NO: 4,
SEQ ID NO: 5 and SEQ ID NO: 4,
SEQ ID NO: 6 and SEQ ID NO: 7,
SEQ ID NO: 8 and SEQ ID NO: 9,
SEQ ID NO: 10 and SEQ ID NO: 11, and
SEQ ID NO: 12 and SEQ ID NO: 13,
whereupon a mRNA transcript of the a isoform of the ER is amplified.

16. A method of generating an expression profile of mRNA transcripts of the α isoform of the ER, wherein said mRNA transcripts comprise alternatively spliced exons, which method comprises:
(i) amplifying a mRNA transcript of the α isoform of the ER according to the method of claim 15,
(ii) contacting the resulting amplicons with a detectably-labeled probe,
(iii) detecting the detectably-labeled probe, and
(iv) quantifying the detectably-labeled probe detected,
whereupon the number of copies of a particular mRNA transcript of the α isoform of the ER is determined.

17. The method of claim 16, which further comprises generating the expression profile of the mRNA transcript of the full-length, wild-type α or β, isoform of the ER, which method comprises:
(i) reverse transcribing said mRNA transcript,
(ii) amplifying the resulting cDNA using a pair of primers comprising SEQ ID NO: 8 and SEQ ID NO: 14, SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, or SEQ ID NO: 26 and SEQ ID NO: 31,
(iii) contacting the resulting amplicons with a detectably-labeled probe,
(iv) detecting the detectably-labeled probe, and
(v) quantifying the detectably-labeled probe detected,
whereupon the number of copies of the full-length, wild-type mRNA transcript of the α or β isoform of the ER is determined.

18. The method of claim 16, wherein the probe is an isolated oligonucleotide about 25 to about 30 nucleotides in length and comprises the sequence of or the sequence that is complementary to any sequence that is found between the sequence to which the targeted primer hybridizes and the sequence to which the partner primer hybridizes, wherein the probe optionally has a fluorochrome at the 5′ end of the probe and a quencher fluorochrome at the 3′ end.

19. The method of claim 41, wherein the probe is selected from the group consisting of:
SEQ ID NO: 32,
SEQ ID NO: 33,
SEQ ID NO: 34,
SEQ ID NO: 35,
SEQ ID NO: 39, and
SEQ ID NO: 42,
wherein SEQ ID NO: 32 is the probe used with the pair of primers SEQ ID NO: 1 and SEQ ID NO: 2,
SEQ ID NO: 33 is the probe used with the pair of primers SEQ ID NO: 3 and SEQ ID NO: 4, SEQ ID NO: 5 and SEQ ID NO: 4, SEQ ID NO: 8 and SEQ ID NO: 9, or SEQ ID NO: 8 and SEQ ID NO: 14,
SEQ ID NO: 34 is the probe used with the pair of primers SEQ ID NO: 6 and SEQ ID NO: 7, or SEQ ID NO: 12 and SEQ ID NO: 13,
SEQ ID NO: 35 is the probe used with the pair of primers SEQ ID NO: 10 and SEQ ID NO: 11,
SEQ ID NO:39 is the probe used with the pair of primers SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, or SEQ ID NO: 26 and SEQ ID NO: 31, and
SEQ ID NO: 42 is the probe used with the pair of primers SEQ ID NO: 40 and SEQ ID NO: 41.

20. The method of claim 35, wherein a standard curve correlating a number of copies of in vitro-transcribed cRNA with a quantity of a detectably-labeled probe detected is used to determine the number of copies of a particular mRNA transcript of the α or β isoform of the ER.

21. The method of claim 35, which further comprises generating the expression profile of a mRNA transcript of GAPDH, which method comprises:
(i) reverse transcribing said mRNA transcript,
(ii) amplifying the resulting cDNA using a pair of primers comprising SEQ ID NO: 40 and SEQ ID NO: 41,
(iii) contacting the resulting amplicons with a detectably-labeled probe,
(iv) detecting the detectably-labeled probe, and
(v) quantifying the detectably-labeled probe detected,
whereupon the number of copies of mRNA transcript of the GAPDH is determined.

22. The method of claim 21, wherein the probe is an isolated oligonucleotide about 25 to about 30 nucleotides in length and comprises the sequence of or the sequence that is complementary to any sequence that is found between the sequence to which the targeted primer hybridizes and the sequence to which the partner primer hybridizes, wherein the probe optionally has a fluorochrome at the 5′ end of the probe and a quencher fluorochrome at the 3′ end.

23. The method of claim 22, wherein the probe is selected from the group consisting of:
SEQ ID NO: 32,
SEQ ID NO: 33,
SEQ ID NO: 34,
SEQ ID NO: 35,
SEQ ID NO: 39, and
SEQ ID NO: 42,
wherein SEQ ID NO: 32 is the probe used with the pair of primers SEQ ID NO: 1 and SEQ ID NO: 2,
SEQ ID NO: 33 is the probe used with the pair of primers SEQ ID NO: 3 and SEQ ID NO: 4, SEQ ID NO: 5 and SEQ ID NO: 4, SEQ ID NO: 8 and SEQ ID NO: 9, or SEQ ID NO: 8 and SEQ ID NO: 14,
SEQ ID NO: 34 is the probe used with the pair of primers SEQ ID NO: 6 and SEQ ID NO: 7, or SEQ ID NO: 12 and SEQ ID NO: 13,
SEQ ID NO: 35 is the probe used with the pair of primers SEQ ID NO: 10 and SEQ ID NO: 11,
SEQ ID NO: 39 is the probe used with the pair of primers SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, or SEQ ID NO: 26 and SEQ ID NO: 31, and,
SEQ ID NO: 42 is the probe used with the pair of primers SEQ ID NO: 40 and SEQ ID NO: 41.

24. The method of claim 21, wherein a standard curve correlating a number of copies of in vitro-transcribed cRNA with a quantity of a detectably-labeled probe detected is used to determine the number of copies of a particular mRNA transcript of the α or β isoform of the ER or the number of copies of the GAPDH mRNA transcript.

25. The method of claim 16, which further comprises generating an expression profile of mRNA transcripts of the β isoform of the ER, wherein said mRNA transcripts comprise alternatively spliced exons, which method comprises:
(i) amplifying a mRNA transcript of the β isoform of the ER according to the method of claim 3,
(ii) contacting the resulting amplicons with a detectably-labeled probe,
(iii) detecting the detectably-labeled probe, and
(iv) quantifying the detectably-labeled probe detected, whereupon the number of copies of a particular mRNA transcript of the β isoform of the ER is determined.

26. The method of claim 25, which further comprises generating the expression profile of the mRNA transcript of the full-length, wild-type α or β isoform of the ER, which method comprises:
(i) reverse transcribing said mRNA transcript,
(ii) amplifying the resulting cDNA using a pair of primers comprising SEQ ID NO: 8 and SEQ ID NO: 14, SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, SEQ ID NO: 26 and SEQ ID NO: 31,
(iii) contacting the resulting amplicons with a detectably-labeled probe,
(iv) detecting the detectably-labeled probe,
(v) quantifying the detectably-labeled probe detected,
whereupon the number of copies of the full-length, wild-type mRNA transcript of the α or β isoform of the ER is determined.

27. The method of claim 25, wherein the targeted primer is selected from the group consisting of:
SEQ ID NO: 15,
SEQ ID NO: 17,
SEQ ID NO: 19,
SEQ ID NO: 22,
SEQ ID NO: 24, and
SEQ ID NO: 25.
wherein the partner primer is selected from the group consisting of:
SEQ ID NO: 16,
SEQ ID NO: 18,
SEQ ID NO: 20,
SEQ ID NO: 21, and
SEQ ID NO: 23, or
wherein the pair of primers comprises a targeted primer and a partner primer selected from the group consisting of:
SEQ ID NO: 15 and SEQ ID NO. 16,
SEQ ID NO: 17 and SEQ ID NO. 18,
SEQ ID NO: 19 and SEQ ID NO. 20,
SEQ ID NO: 22 and SEQ ID NO. 21,
SEQ ID NO: 24 and SEQ ID NO. 23, and
SEQ ID NO: 25 and SEQ ID NO. 21.

28. The method of any of claim 25, wherein the probe is an isolated oligonucleotide about 25 to about 30 nucleotides in length and comprises the sequence of or the sequence that is complementary to any sequence that is found between the sequence to which the targeted primer hybridizes and the sequence to which the partner primer hybridizes, wherein the probe comprises a fluorochrome at the 5′ end of the probe and a quencher fluorochrome at the 3′ end of the probe.

29. The method of claim 28, wherein the probe is selected from the group consisting of:
SEQ ID NO: 32,
SEQ ID NO: 33,
SEQ ID NO: 34,
SEQ ID NO: 35,
SEQ ID NO:36,
SEQ ID NO: 37,
SEQ ID NO: 38,
SEQ ID NO: 39 and
SEQ ID NO: 42,
wherein SEQ ID NO: 32 is the probe used with the pair of primers SEQ ID NO: 1 and SEQ ID NO: 2,
SEQ ID NO: 33 is the probe used with the pair of primers SEQ ID NO: 3 and SEQ ID NO: 4, SEQ ID NO: 5 and SEQ ID NO: 4, SEQ ID NO: 8 and SEQ ID NO: 9, or SEQ ID NO: 8 and SEQ ID NO: 14,
SEQ ID NO: 34 is the probe used with the pair of primers SEQ ID NO: 6 and SEQ ID NO: 7, or SEQ ID NO: 12 and SEQ ID NO: 13,
SEQ ID NO: 35 is the probe used with the pair of primers SEQ ID NO: 10 and SEQ ID NO: 11,
SEQ ID NO:36 is the probe used with the pair of primers SEQ ID NO: 15 and SEQ ID NO: 16,
SEQ ID NO: 37 is the probe used with the pair of primers SEQ ID NO: 17 and SEQ ID NO: 18, SEQ ID NO: 21 and SEQ ID NO: 22, SEQ ID NO: 21 and SEQ ID NO: 25,
SEQ ID NO: 38 is the probe used with the pair of primers SEQ ID NO: 19 and SEQ ID NO: 20, or SEQ ID NO: 23 and SEQ ID NO: 24,
SEQ ID NO: 39 is the probe used with the primers SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, or SEQ ID NO: 26 and SEQ ID NO: 31, and,
SEQ ID NO: 42 is the probed used with the pair of primers of SEQ ID NO: 40 and SEQ ID NO: 41.

30. The method of any of claim 25, wherein a standard curve correlating a number of copies of in vitro-transcribed cRNA with a quantity of a detectably-labeled probe detected is used to determine the number of copies of a particular mRNA transcript of the β isoform of the ER or the number of copies of the mRNA transcript of the GAPDH.

31. The method of any of claim 25, which further comprises generating the expression profile of a mRNA transcript of GAPDH, which method comprises:
(i) reverse transcribing said mRNA transcript,
(ii) amplifying the resulting cDNA using a pair of primers comprising SEQ ID NO: 40 and SEQ ID NO: 41,
(iii) contacting the resulting amplicons with a detectably-labeled probe,
(iv) detecting the detectably-labeled probe, and
(v) quantifying the detectably-labeled probe detected,
whereupon the number of copies of mRNA transcript of the GAPDH is determined.

32. The method of claim 31, wherein the probe is an isolated oligonucleotide about 25 to about 30 nucleotides in length and comprises the sequence of or the sequence that is complementary to any sequence that is found between the sequence to which the targeted primer hybridizes and the sequence to which the partner primer hybridizes, wherein the probe comprises a fluorochrome at the 5′ end of the probe and a quencher fluorochrome at the 3′ end of the probe.

33. The method of claim 32, wherein the probe is selected from the group consisting of:
SEQ ID NO: 32,
SEQ ID NO: 33,
SEQ ID NO: 34,
SEQ ID NO: 35,
SEQ ID NO:36,
SEQ ID NO: 37,
SEQ ID NO: 38,
SEQ ID NO: 39 and
SEQ ID NO: 42,
wherein SEQ ID NO: 32 is the probe used with the pair of primers SEQ ID NO: 1 and SEQ ID NO: 2,
SEQ ID NO: 33 is the probe used with the pair of primers SEQ ID NO: 3 and SEQ ID NO: 4, SEQ ID NO: 5 and SEQ ID NO: 4, SEQ ID NO: 8 and SEQ ID NO: 9, or SEQ ID NO: 8 and SEQ ID NO: 14,
SEQ ID NO: 34 is the probe used with the pair of primers SEQ ID NO: 6 and SEQ ID NO: 7, or SEQ ID NO: 12 and SEQ ID NO: 13,
SEQ ID NO: 35 is the probe used with the pair of primers SEQ ID NO: 10 and SEQ ID NO: 11,
SEQ ID NO:36 is the probe used with the pair of primers SEQ ID NO: 15 and SEQ ID NO: 16,
SEQ ID NO: 37 is the probe used with the pair of primers SEQ ID NO: 17 and SEQ ID NO: 18, SEQ ID NO: 21 and SEQ ID NO: 22, SEQ ID NO: 21 and SEQ ID NO: 25,
SEQ ID NO: 38 is the probe used with the pair of primers SEQ ID NO: 19 and SEQ ID NO: 20, or SEQ ID NO: 23 and SEQ ID NO: 24,
SEQ ID NO: 39 is the probe used with the primers SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, or SEQ ID NO: 26 and SEQ ID NO: 31, and,
SEQ ID NO:42 is the probed used with the pair of primers of SEQ ID NO: 40 and SEQ ID NO: 41.

34. The method of claim 31, wherein a standard curve correlating a number of copies of in vitro-transcribed cRNA with a quantity of a detectably-labeled probe detected is used to determine the number of copies of a particular mRNA transcript of the β isoform of the ER or the number of copies of the mRNA transcript of the GAPDH.

35. A method of generating an expression profile of a mRNA transcript of full-length, wild-type α or β isoform of the ER, which method comprises:
(i) reverse-transcribing said mRNA transcript,
(ii) amplifying the resulting cDNA using a pair of primers comprising SEQ ID NO: 8 and SEQ ID NO: 14, SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, or SEQ ID NO: 26 and SEQ ID NO: 31,
(iii) contacting the resulting amplicons with a detectably-labeled probe,
(iv) detecting the detactably-labeled probe, and
(v) quantifying the detectably-labeled probed detected,
whereupon the number of copies of the full-length, wild-type mRNA transcript of the α or β isoform of the ER is determined.

36. The method of claim 35, which further comprises generating the expression profile of a mRNA transcript of GAPDH, which method comprises:
(i) reverse transcribing said mRNA transcript,
(ii) amplifying the resulting cDNA using a pair of primers comprising SEQ ID NO: 40 and SEQ ID NO: 41,
(iii) contacting the resulting amplicons with a detectably-labeled probe,
(iv) detecting the detectably-labeled probe, and
(v) quantifying the detectably-labeled probe detected,
whereupon the number of copies of mRNA transcript of the GAPDH is determined.

37. The method of claim 35, wherein the probe is an isolated oligonucleotide about 25 to about 30 nucleotides in length and comprises the sequence of or the sequence that is complementary to any sequence that is found between the sequence to which the targeted primer hybridizes and the sequence to which the partner primer hybridizes, wherein the probe optionally comprises a fluorochrome at the 5′ end of the probe and a quencher fluorochrome at the 3′ end of the probe.

38. The method of claim 37, wherein the probe is selected from the group consisting of:
SEQ ID NO: 33,
SEQ ID NO: 39, and
SEQ ID NO: 41,
wherein SEQ ID NO: 33 is the probe used with the pair of primers SEQ ID NO: 8 and SEQ ID NO: 14,
SEQ ID NO: 39 is the probe used with the pair of primers SEQ ID NO: 26 and SEQ ID NO: 27, SEQ ID NO: 26 and SEQ ID NO: 28, SEQ ID NO: 26 and SEQ ID NO: 29, SEQ ID NO: 26 and SEQ ID NO: 30, or SEQ ID NO: 26 and SEQ ID NO: 31, and,
SEQ ID NO: 42 is the probe used with the pair of primers SEQ ID NO: 40 and SEQ ID NO: 41.

39. The method of claim 35, wherein a standard curve correlating a number of copies of in vitro-transcribed cRNA with a quantity of a detectably-labeled probe detected is used to determine the number of copies of a particular mRNA transcript of the α or β isoform of the ER or the number of copies of the mRNA transcript of the GAPDH.

40. A method of generating a standard curve, wherein the standard curve correlates a number of copies of in vitro-transcribed cRNA with a quantity of a detectably-labeled probe detected, which method comprises:
(i) in vitro transcribing RNA from a DNA plasmid, wherein said DNA plasmid encodes GAPDH, the wild-type protein of the α or β ER, an alternatively-spliced isoform of the α or β ER, or a fragment of either of the foregoing, wherein, when the fragment is that of an alternatively spliced isoform of the α or β ER, the fragment comprises the alternative splice junction,
(ii) reverse-transcribing the resulting cRNA,
(iii) amplifying the resulting cDNA using the pair of primers that is used to amplify GAPDH, the wild-type protein of the α or β ER, the alternatively-spliced isoform of the α or β ER, or a fragment of either of the foregoing encoded in the DNA plasmid of step (i) selected from the group consisting of:
SEQ ID NO: 40 and SEQ ID NO: 41,
SEQ ID NO: 1 and SEQ ID NO: 2,
SEQ ID NO: 3 and SEQ ID NO: 4,
SEQ ID NO: 5 and SEQ ID NO: 4,
SEQ ID NO: 6 and SEQ ID NO: 7,
SEQ ID NO: 8 and SEQ ID NO: 9,
SEQ ID NO: 10 and SEQ ID NO: 11,
SEQ ID NO: 12 and SEQ ID NO: 13,
SEQ ID NO: 8 and SEQ ID NO: 14,
SEQ ID NO: 15 and SEQ ID NO: 16,
SEQ ID NO: 17 and SEQ ID NO: 18,
SEQ ID NO: 19 and SEQ ID NO: 20,
SEQ ID NO: 21 and SEQ ID NO: 22,
SEQ ID NO: 23 and SEQ ID NO: 24,
SEQ ID NO: 21 and SEQ ID NO: 25,
SEQ ID NO: 26 and SEQ ID NO: 27,
SEQ ID NO: 26 and SEQ ID NO: 28,
SEQ ID NO: 26 and SEQ ID NO: 29,
SEQ ID NO: 26 and SEQ ID NO: 30, and
SEQ ID NO: 26 and SEQ ID NO: 31,
(iv) contacting the resulting amplified products with a detectably-labeled probe,
(v) detecting the detectably-labeled probe,
(vi) quantifying the detectably-labeled probe detected, and
(vii) repeating steps (ii)-(vi) at least 6 times, wherein the amount of cRNA used in step (ii) is different each time,
whereupon a standard curve correlating a number of copies of in vitro-transcribed cRNA with a quantity of detectably-labeled probe detected is generated.

41. A method of generating cRNA of a GAPDH or of an α or β isoform of the ER, which method comprises:
(i) cloning a fragment of a coding sequence of the GAPDH or of the α or β isoform of the ER into a pCR®2.1-TOPO vector, wherein the fragment is at least about 500 base pairs in length,
(ii) linearizing the resulting vector with BamHI,
(iii) using T7 RNA polymerase to generate cRNA, and
(iv) cleaving the vector with DNA-free™DNAse,
whereupon cRNA for the GAPDH or the α or β isoform of the ER is generated.

42. A method of generating cRNA of an ERα5Δ isoform of the ER, which method comprises:
(i) cloning a fragment of a coding sequence of the ERα5Δ isoform into a pCR®2.1-TOPO vector, wherein the fragment is at least about 500 base pairs in length,
(ii) digesting the resulting vector with EcoRI to obtain a fragment,
(iii) subcloning the fragment into a pSG vector,
(iv) linearizing the resulting vector with BglII,
(v) using T7 RNA polymerase to generate cRNA, and
(vi) cleaving the vector with DNA-free™DNAse,
whereupon cRNA for the ERα5Δ isoform of the ER is generated.

43. A method of generating a standard curve, wherein the standard curve correlates a number of copies of in vitro-transcribed cRNA with a quantity of a detectably-labeled probe detected, which method comprises:
(i) generating cRNA of GAPDH, the wild-type protein of the α or β ER, an alternatively-spliced isoform of the α or β ER, or a fragment of either of the foregoing, wherein, when the fragment is that of an alternatively spliced isoform of the β or β ER, the fragment comprises the alternative splice junction, in accordance with claim 41,
(ii) reverse-transcribing the resulting cRNA,
(iii) amplifying the resulting cDNA using the pair of primers that are used to amplify the wild-type protein of the α or β ER, the alternatively-spliced isoform of the α or β ER, or a fragment of either of the foregoing encoded in the DNA plasmid of step (i) selected from the group consisting of:
SEQ ID NO: 40 and SEQ ID NO: 41,
SEQ ID NO: 1 and SEQ ID NO: 2,
SEQ ID NO: 3 and SEQ ID NO: 4,
SEQ ID NO: 5 and SEQ ID NO: 4,
SEQ ID NO: 6 and SEQ ID NO: 7,
SEQ ID NO: 8 and SEQ ID NO: 9,
SEQ ID NO: 10 and SEQ ID NO: 11,
SEQ ID NO: 12 and SEQ ID NO: 13,
SEQ ID NO: 8 and SEQ ID NO: 14,
SEQ ID NO: 15 and SEQ ID NO: 16,
SEQ ID NO: 17 and SEQ ID NO: 18,
SEQ ID NO: 19 and SEQ ID NO: 20,
SEQ ID NO: 21 and SEQ ID NO: 22,
SEQ ID NO: 23 and SEQ ID NO: 24,
SEQ ID NO: 21 and SEQ ID NO: 25,
SEQ ID NO: 26 and SEQ ID NO: 27,
SEQ ID NO: 26 and SEQ ID NO: 28,
SEQ ID NO: 26 and SEQ ID NO: 29,
SEQ ID NO: 26 and SEQ ID NO: 30, and
SEQ ID NO: 26 and SEQ ID NO: 31,
(iv) contacting the resulting amplified products with a detectably-labeled probe,
(v) detecting the detectably-labeled probe,
(vi) quantifying the detectably-labeled probe detected, and
(vii) repeating steps (ii)-(vi) at least 6 times, wherein the amount of cRNA used in step (ii) is different each time,
whereupon a standard curve correlating a number of copies of in vitro-transcribed cRNA with a quantity of detectably-labeled probe detected is generated.

44. A method of generating a standard curve, wherein the standard curve correlates a number of copies of in vitro-transcribed cRNA with a quantity of a detectably-labeled probe detected, which method comprises:
(i) generating cRNA of GAPDH, the wild-type protein of the α or β ER, an alternatively-spliced isoform of the α or β ER, or a fragment of either of the foregoing, wherein, when the fragment is that of an alternatively spliced isoform of the α or β ER, the fragment comprises the alternative splice junction, in accordance with claim 42,
(ii) reverse-transcribing the resulting cRNA,
(iii) amplifying the resulting cDNA using the pair of primers that are used to amplify the wild-type protein of the α or β ER, the alternatively-spliced isoform of the α or β ER, or a fragment of either of the foregoing encoded in the DNA plasmid of step (i) selected from the group consisting of:
SEQ ID NO: 40 and SEQ ID NO: 41,
SEQ ID NO: 1 and SEQ ID NO: 2,
SEQ ID NO: 3 and SEQ ID NO: 4,
SEQ ID NO: 5 and SEQ ID NO: 4,
SEQ ID NO: 6 and SEQ ID NO: 7,
SEQ ID NO: 8 and SEQ ID NO: 9,
SEQ ID NO: 10 and SEQ ID NO: 11,
SEQ ID NO: 12 and SEQ ID NO: 13,
SEQ ID NO: 8 and SEQ ID NO: 14,
SEQ ID NO: 15 and SEQ ID NO: 16,
SEQ ID NO: 17 and SEQ ID NO: 18,
SEQ ID NO: 19 and SEQ ID NO: 20,
SEQ ID NO: 21 and SEQ ID NO: 22,
SEQ ID NO: 23 and SEQ ID NO: 24,
SEQ ID NO: 21 and SEQ ID NO: 25,
SEQ ID NO: 26 and SEQ ID NO: 27,
SEQ ID NO: 26 and SEQ ID NO: 28,
SEQ ID NO: 26 and SEQ ID NO: 29,
SEQ ID NO: 26 and SEQ ID NO: 30, and
SEQ ID NO: 26 and SEQ ID NO: 31,
(iv) contacting the resulting amplified products with a detectably-labeled probe,
(v) detecting the detectably-labeled probe,
(vi) quantifying the detectably-labeled probe detected, and
(vii) repeating steps (ii)-(vi) at least 6 times, wherein the amount of cRNA used in step (ii) is different each time,
whereupon a standard curve correlating a number of copies of in vitro-transcribed cRNA with a quantity of detectably-labeled probe detected is generated.

45. A method of determining breast cancer or a risk for breast cancer in a mammal, which method comprises comparing the expression profile of an ER-containing sample of the mammal with the expression profile of a control standard, wherein said expression profile has been obtained in accordance with claim 5 wherein a difference in the expression profile of the mammal in comparison to the expression profile of the control standard is indicative of breast cancer or risk for breast cancer in the mammal.

46. A method of determining breast cancer or a risk for breast cancer in a mammal, which method comprises comparing the expression profile of an ER-containing sample of the mammal with the expression profile of a control standard, wherein said expression profile has been obtained in accordance with claim 16 wherein a difference in the expression profile of the mammal in comparison to the expression profile of the control standard is indicative of breast cancer or risk for breast cancer in the mammal

47. A method of determining breast cancer or a risk for breast cancer in a mammal, which method comprises comparing the expression profile of an ER-containing sample of the mammal with the expression profile of a control standard, wherein said expression profile has been obtained in accordance with claim 35 wherein a difference in the expression profile of the mammal in comparison to the expression profile of the control standard is indicative of breast cancer or risk for breast cancer in the mammal.

48. A method of prognosticating response of a mammal to a therapeutic treatment of breast cancer in the mammal, which method comprises comparing the expression profiles of ER-containing samples of the mammal obtained over the course of therapeutic treatment of the mammal, wherein said expression profiles have been obtained in accordance with the method of claim 5.

49. A method of prognosticating response of a mammal to a therapeutic treatment of breast cancer in the mammal, which method comprises comparing the expression profiles of ER-containing samples of the mammal obtained over the course of therapeutic treatment of the mammal, wherein said expression profiles have been obtained in accordance with the method of claim 16.

50. A method of prognosticating response of a mammal to a therapeutic treatment of breast cancer in the mammal, which method comprises comparing the expression profiles of ER-containing samples of the mammal obtained over the course of therapeutic treatment of the mammal, wherein said expression profiles have been obtained in accordance with the method of claim 35.

51. A method of determining a hyperproliferative condition or a predisposition to a hyperproliferative condition in a mammal, which method comprises comparing the expression profile of an ER-containing sample of the mammal with the expression profile of a control standard, wherein said expression profiles have been obtained in accordance with claim 5, wherein a difference in the expression profile of the mammal in comparison to the expression profile of the control standard is indicative of a hyperproliferative condition or a predisposition to a hyperproliferative condition in the mammal.

52. A method of determining a hyperproliferative condition or a predisposition to a hyperproliferative condition in a mammal, which method comprises comparing the expression profile of an ER-containing sample of the mammal with the expression profile of a control standard, wherein said expression profiles have been obtained in accordance with claim 16, wherein a difference in the expression profile of the mammal in comparison to the expression profile of the control standard is indicative of a hyperproliferative condition or a predisposition to a hyperproliferative condition in the mammal.

53. A method of determining a hyperproliferative condition or a predisposition to a hyperproliferative condition in a mammal, which method comprises comparing the expression profile of an ER-containing sample of the mammal with the expression profile of a control standard, wherein said expression profiles have been obtained in accordance with claim 35, wherein a difference in the expression profile of the mammal in comparison to the expression profile of the control standard is indicative of a hyperproliferative condition or a predisposition to a hyperproliferative condition in the mammal.

54. A method of screening candidate therapeutic treatments for an ER-sensitive condition, which method comprises comparing the expression profile of an ER-containing sample before treatment with the expression profile of the ER-containing sample after treatment, wherein said expression profiles have been obtained in accordance with claim 9.

55. A method of screening candidate therapeutic treatments for an ER-sensitive condition, which method comprises comparing the expression profile of an ER-containing sample before treatment with the expression profile of the ER-containing sample after treatment, wherein said expression profiles have been obtained in accordance with claim 16.

56. A method of screening candidate therapeutic treatments for an ER-sensitive condition, which method comprises comparing the expression profile of an ER-containing sample before treatment with the expression profile of the ER-containing sample after treatment, wherein said expression profiles have been obtained in accordance with claim 35.
